(19) United States
(12) Patent Application Publication
(10) Pub. No.: US 2004/0023362 A1
(43) Pub. Date: Feb. 5, 2004

(54) Degradation of polycyclic aromatic hydrocarbons by microorganisms

(76) Inventors: Grant Stanley, Victoria (AU); Margaret Britz, Victoria (AU); Sudarat Boonchan, Choncuri (TH)

Correspondence Address: GREER, BURNS & CRAIN, 300 S WACKER DR, 25TH FLOOR, CHICAGO, IL, 60606, US

(21) Appl. No.: 10/220,108

(22) PCT Filed: Feb. 28, 2001
(86) PCT No.: PCT/AU01/00202

(30) Foreign Application Priority Data
Feb. 28, 2000 (AU) PQ 5886

(51) Int. Cl. C12N 1/14; C12S 1/00; B09B 3/00

(52) U.S. Cl. 435/262.5; 435/254.3

(57) ABSTRACT

The invention relates to isolated microorganisms useful for the biodegradation of polycylic aromatic hydrocarbons (PAHs). Specifically, the invention relates to isolated Stenotrophomonas maltophilia VUN 10,010, Pseudomonas fluorescens VUN 10,011, Burkholderia sp. VUN 10,013 and Penicillium janthinellum. The invention further relates to a bacterial consortium having the ability to degrade PAHs.

This invention relates to methods and compositions for degradation of polycyclic aromatic hydrocarbons. In particular, the invention relates to microorganisms which are able to degrade polycyclic aromatic hydrocarbons, and to methods and compositions which utilise these microorganisms. In a preferred embodiment, the invention relates to a composition which is able to degrade polycyclic aromatic hydrocarbons completely to carbon dioxide and water. The microorganisms, compositions and methods of the invention are useful in the bioremediation of materials contaminated with polycyclic aromatic hydrocarbons, such as soils, sediments and liquid effluents.

### BACKGROUND OF THE INVENTION

Polycyclic aromatic hydrocarbons (PAHs) are hydrophobic organic compounds which are commonly found in the environment through the disposal of coal processing wastes, petroleum sludges, asphalt, creosote and other wood preservative wastes (Wilson and Jones, 1993). The decontamination of PAH-polluted sites is of major importance because many PAH compounds are either known or suspected carcinogens and mutagens (Wright, 1980). Most low molecular weight PAHs are biodegradable in the presence of suitable microbial populations, and a number of bioremediation programs have had some success in the decontamination of PAH-contaminated sites. However, the extent and rate of PAH biodegradation are restricted by the limited bioavailability of these compounds, which is due to their low aqueous solubilities and strong adsorptive capacity to soil and sediments (McElroy et al, 1989). Indeed, the mass transfer rate of naphthalene and phenanthrene into the aqueous phase was shown to be the rate-limiting step in their biodegradation (Bury and Miller, 1993; Grimberg et al, 1996; Volkering et al, 1995).

Human exploitation of fossil fuel reserves and the production of novel synthetic compounds have introduced many pollutants into the environment. In recent years there has been an increasing awareness and concern regarding the disposal and accumulation of polycyclic aromatic hydrocarbons (PAHs) in the ecosystem. Many low molecular weight PAHs are acutely toxic, with some having effects on the reproduction and mortality rates of aquatic animals, and most high molecular weight PAHs are mutagenic and carcinogenic. Owing to their hydrophobic nature, most PAHs in aquatic and terrestrial ecosystems become associated with particulates and are bound to soil and sediments, rendering them less available for biological uptake. There is also a potential for the bioaccumulation of PAHs into food chains (Morehead et al, 1986). Because of their toxicity, carcinogenicity, recalcitrance and ubiquitous distribution, the U.S Environment Protection Agency has listed sixteen PAH compounds as priority pollutants to be monitored in industrial effluents (Keith and Telliard, 1979).

Microbial degradation represents the major method responsible for the ecological recovery of PAH-contaminated sites (Cerniglia, 1992). However, the success of bioremediation projects has been limited by the failure to remove high molecular weight PAHs (Wilson and Jones, 1993). The recalcitrance of high molecular weight PAHs to microbial degradation has led to a recent research focus on evaluating a wide phylogenetic spectrum of microorganisms for the degradation of these compounds. A diverse group of bacteria and fungi with the ability to partially degrade, cometabolically oxidize or mineralise high molecular weight PAHs to detoxified chemical products have so far been identified.

The highest molecular weight PAHs to be mineralised as a sole source of carbon and energy by bacterial isolates reported in the literature to date contain four-benzene rings, such as pyrene and chrysene. The species involved include Rhodococcus sp. (Walter et al, 1991), Burkholderia cepacia (Juhasz et al, 1997; Juhasz, et al, 1996), Mycobacterium sp. (Bouchez et al, 1995; Heitkamp et al, 1988; Kästner et al, 1994), Alcaligenes denitrificans (Weissenfels et al, 1990) and Sphingomonas paucimobilis (Mueller et al, 1990; Ye et al, 1996). Many of these bacteria are also able to degrade five-benzene ring PAHs, but in most cases degradation goes no further than the production of oxidised PAH compounds.

In contrast to bacteria, fungi generally do not utilize PAHs as their sole carbon and energy source, but transform these compounds co-metabolically to detoxified products (Sutherland, 1992). A diverse group of lignolytic and non-lignolytic fungi are able to oxidize PAHs. The most extensive studies have focused on white rot fungi, such as Phanerochaete chrysosporium (Barclay et al, 1995; Brodkorb and Legge, 1992; Bumpus, 1989), Pleurotus ostreatus (Bezalel et al, 1996; Vyas et al, 1994) and Trametes versicolor (Anderson and Henrysson, 1996; Collins et al, 1996; Vyas et al, 1994). These fungi are able to degrade the potent carcinogen benzo[a]pyrene and to detoxify PAH-polluted soils and sediments due to their production of extracellular lignin-degrading enzymes. Non-lignolytic fungi may also degrade high molecular weight PAHs. For example, Cunninghamella elegans, Penicillium janthinellum and Syncephalastrum sp. can transform a variety of PAHs, including pyrene, chrysene and benzo[a]pyrene, to polar metabolites (Kiehlmann et al, 1996; Launen et al, 1995; Pothuluri et al, 1994; Wunder et al, 1997). Only a few reports have described benzo[a]pyrene mineralisation in pure microbial cultures. These pertain to the co-metabolic mineralisation of benzo[a]pyrene by Phanerochaete spp. in a medium containing benzo[a]pyrene and at least one other carbon substrate (Barclay et al, 1995; Bogan and Lamar, 1996; Bumpus et al, 1985; Sanglard et al, 1986).

There is only one report which describes the mineralisation of benzo[a]pyrene by bacteria. In this case, benzo[a]pyrene was mineralised by a resting cell suspension of a Sphingomonas paucimobilis strain, but the organism could not use this PAH as a sole carbon and energy source (Ye et al, 1996). The fact that no single microorganism has yet been isolated which is capable of growth on, and mineralisation of, PAHs containing five or more benzene rings as a sole carbon and energy source suggests that mineralisation of these compounds in nature largely depends upon the cooperative metabolic activities of mixed microbial populations.

One way to enhance the solubility of PAHs is to apply mobilizing agents such as surfactants (Ganeshlingam et al, 1994; Scheibenbogen et al, 1994). Due to their amphiphilic nature, surfactant molecules may dissolve in water as monomers, adsorb at an interface, or be incorporated with other surfactant molecules as part of a micelle (Edward et al, 1991); the surfactant concentration at which micelles are formed initially is termed the critical micelle concentration (CMC). Micelle formation allows PAH compounds to partition within the hydrophobic core of the micelle, leading to an increase in the apparent solubility of these compounds in the liquid phase (Edwards et al, 1991; Rouse et al, 1994). Although PAHs in the micellar phase are reportedly not directly available for microbial degradation, the dissolution rate of some low molecular weight PAHs into the aqueous phase was enhanced when surfactant micelles were present (Edward et al, 1991; Grimberg et al, 1996; Liu et al, 1995; Volkering et al, 1995).

When surfactants have been used in attempts to accelerate the biodegradation of PAHs, variable impacts have been reported; some authors noted improved degradation rates (Grimberg et al, 1996; Volkering et al, 1995), while others reported either no impact (Fought et al, 1989; Ghosh et al, 1995) or inhibitory effects (Deschenes et al, 1995; Laha and Luthy, 1991). This suggests that a variety of factors influence biodegradation, including surfactant type and concentration, the substrate mixture and the microbial species involved, and that success for any given system cannot be confidently predicted.

To date, naphthalene and phenanthrene have been the most commonly used model substrates for evaluating the impact of surfactants on biodegradation (Edwards et al, 1991; Grimberg et al, 1994; Volkering et al, 1995). Higher molecular weight PAH compounds containing four-, five- and seven-benzene rings are often a significant proportion of most PAH-contaminated sites, and these compounds are the most recalcitrant in terms of bioremediation, due to their very low water solubility and high adsorption to particulate surfaces (Mahaffey et al, 1991; Shuttleworth and Cerniglia, 1995). Weathered contaminated soils are typically low in the lighter PAH fractions which are removed by processes such as volatilization and biodegradation; the remaining heavier fractions may form a matrix which has an extremely low water solubility and high recalcitrance.

Mobilization of high molecular weight PAHs from these soils by ex situ and in situ soil flushing with surfactant solutions may prove useful in treating weathered contaminated sites (Churchill et al, 1995; Joshi and Lee, 1996; Yeom et al, 1996). However, the resulting leachates contain the surfactant plus a PAH mixture with a high proportion of high molecular weight PAHs, and this must then be treated separately (Yeom et al, 1996). There is scant information on the treatment of such leachates using microorganisms, because of the lack of microbial isolates which can degrade higher molecular weight PAHs. Tiehm (1994) reported using a pyrene-degrading Mycobacterium sp. in the biodegradation of fluoranthene and pyrene in a PAH mixture that also contained several three-ring PAHs, where the rate of degradation was improved by the addition of nonionic surfactant Sapogenet T-300. This suggests that surfactant addition may also enhance the rates of biodegradation the more recalcitrant five- and seven-ring compounds, such as benzo[a]pyrene and coronene, if microbial isolates capable of degrading these PAHs are used.

We have isolated a novel co-culture, comprising Penicillium janthinellum VUO 10,201 and bacterial consortium VUN 10,009, which mineralises and grows on benzo[a]pyrene as a sole carbon and energy source. We have also isolated a novel bacterium, Stenotrophomonas maltophilia VUN10,010 from a different PAH-contaminated site to VUO 10,201. Benzo[a]pyrene mineralisation rates were improved when P. janthinellum VUO 10,201 was co-cultured with Stenotrophomonas maltophilia VUN 10,010. Exceptional benzo[a]pyrene mineralisation by fungal-bacterial co-cultures was observed in liquid culture and in authentic PAH-contaminated soil, compared to axenic cultures of these organisms and of the indigenous microflora. We have also evaluated different surfactants for their utility in improving the biodegradation of four-, five- and seven-ring PAHs by the organisms of the invention.

### SUMMARY OF THE INVENTION

In a first aspect, the invention provides an isolated culture of a microorganism having the ability to degrade high molecular weight polycyclic aromatic hydrocarbons (PAHs), said microorganism being selected from the group consisting of Stenotrophomonas maltophilia VUN 10,010 or VUN 10,012, Pseudomonas fluorescens VUN 10,011, Burkholderia sp. VUN 10,013, Penicillium janthinellum, and bacterial consortium VUN 10,009.

Preferably the microorganism is able to degrade PAHs of three to eight benzene rings. More preferably the PAH has more than four benzene rings. More preferably the PAH has five or more benzene rings. Most preferably the PAH has five to ten benzene rings.

In one particularly preferred embodiment, the Penicillium janthinellum is the strain designated VUO 10,201, a sample of which was deposited under the provisions of the Budapest Treaty in the Australian Government Analytical Laboratories on Oct. 21, 1998under Accession No. NM 98/09532, or a mutant or derivative thereof having the ability to degrade PAHs. In a second preferred embodiment, the Stenotrophomonas maltophilia is selected from the group consisting of strains VUN 10,010 and VUN 10,012, samples of which were deposited under the provisions of the Budapest Treaty in the Australian Government Analytical Laboratories on Oct. 21, 1998 under Accession No. NM 98/09528 and No. NM 98/09530, or a mutant or derivative thereof having the ability to degrade PAHs.

In a third preferred embodiment, the microorganism is bacterial consortium VUN 10,009, a sample of which was deposited under the provisions of the Budapest Treaty in the Australian Government Analytical Laboratories on Oct. 21, 1998 under Accession No. NM 98/09527, or a mutant or derivative thereof having the ability to degrade PAHs, or the pure isolate of Burkholderia sp VUN 10,013 obtained therefrom, a sample of which was deposited under the provisions of the Budapest Treaty in the Australian Government Analytical Laboratories on Oct. 21, 1998 under Accession No. NM 98/09531.

In a fourth preferred embodiment, the microorganism is Pseudomonas fluorescens VUN 10,011, a sample of which was deposited at under the provisions of the Budapest Treaty in the Australian Government Analytical Laboratories on Oct. 21, 1998 under Accession No. NM 98/09529.

Mutants or derivatives of the specific microorganisms disclosed herein can be generated and selected using methods well known in the art, for example exposure to ionizing radiation, ultraviolet radiation, or chemical mutagens, and selection for ability to grow in the presence of high concentration of PAHs. By using such methods, strains of even greater activity than those described herein can be generated.

In a particularly preferred form, the microorganism of the invention is able to utilise one or more PAHs as sole carbon and energy source. In an even more preferred form, the microorganism is able to mineralise one or more PAHs, ie. to convert the PAH completely to carbon dioxide and water. It is expected that the microorganisms of the invention are able also to degrade PAHs which are substituted with one or more groups selected from the group consisting of alkali, halogen and nitro. It is also expected that the microorganisms can degrade other organic compounds such as halogenated aliphatic compounds. We have shown that the microorganisms of the invention can degrade compounds such as phenol, salicylate, benzene, toluene and xylene, halogenated single ring aromatic compounds, and non-halogenated aromatic compounds.

In a second aspect, the invention provides an agent for degrading PAHs, comprising one or more microorganisms selected from the group consisting of Penicillium janthinellum VUN 10,201, bacterial consortium VUN 10,009, Stenotrophomonas maltophilia VUN 10,010, Pseudomonas fluorescens VUN 10,011 and Burkholderia sp. VUN 10,013, as described above. Preferably the agent comprises a co-culture of Penicillium janthinellum VUO 10,201 and bacterial consortium VUN 10,009, optionally further comprising an additional microorganism selected from the group consisting of Stenotrophomonas maltophilia VUN 10,010 or VUN 10,012, Pseudomonas fluorescens VUN 10,011 and Burkholderia sp. VUN 10,013. More preferably the agent comprises all three of these microorganisms.

Most preferably the agent comprises a co-culture of Penicillium janthinellum VUO 10,201 and bacterial consortium VUN 10,009 or Stenotrophomonas maltophilia VUN 10,010.

The agent may further comprise one or more carriers or excipients. Consequently the invention additionally provides compositions comprising the microorganisms of the invention. The nature of the carrier or excipient will depend on the particular purpose for which the agent is to be used, and on the site to which it is to be applied. However, it is contemplated that the agent may be provided in the form of sprays, foams, liquids and the like. The agent may be provided on agar slants or frozen (at −20° C. or −80° C.) in liquid medium containing a cryoprotectant, in a lyophilized form and stored in a vacuum under various conditions, or as a liquid culture at room temperature.

We have found that non-ionic surfactants increase the ability of S. maltophilia VUN 10,010, or of co-cultures of Penicillium janthinellum VUO 10,201 and bacterial consortium VUN 10,009, to degrade high molecular weight PAHs, and therefore in one preferred form the agents and compositions of the invention comprise a non-ionic surfactant. Preferably the surfactant is selected from the group consisting of Brij 35, Igepal CA-630, Triton X-100, Tergitol NP-10 and Tyloxapol. More preferably the surfactant is Brij 35 or Tergitol NP-10. The amount of surfactant to be used will depend on the nature and level of contamination of the site to be treated, and can readily be assessed by trial and error experimentation.

In a third aspect, the invention provides a method for degrading PAHs, comprising the step of exposing the PAHs to a microorganism or agent of the invention.

The methods of the invention are suitable for bioremediation of soils, sediments or liquids contaminated with PAHs. These methods are also applicable to reducing the mutagenic capacity of PAHs. Preferably the PAH comprises one or more of pyrene, chrysene, benz[a]anthracene, benzo[a]pyrene and dibenz[a,h]anthracene.

By utilizing the methods of the invention, the PAHs will be at least partly degraded, and thus will be converted to a less toxic and less mutagenic form. In a preferred embodiment, one or more of the PAHs is converted completely to carbon dioxide and water.

For the purposes of this specification, it will be clearly understood that the term “mineralisation” with reference to PAH means conversion of the PAH to carbon dioxide and water

For the purposes of this specification it will also be clearly understood that the word “comprising” means “including but not limited to”, and that the word “comprises” has a corresponding meaning.

### BRIEF DESCRIPTION OF THE FIGURES

FIG. 1 shows degradation of pyrene, benzo[a]pyrene and dibenz[a,h]anthracene in BSM by P. janthinellum VUO 10,201 (□), bacterial consortium VUN 10,009 (O) or co-culture A (▴). PAH-BSM media inoculated with a killed co-culture A (▾) is also shown. Culture medium comprised BSM and a single PAH. Bacterial growth is shown as an estimated cell number determined in axenic cultures (O) and co-culture A (●). Fungal dry weight is shown for axenic cultures (□) and co-culture A (▪).

Co-culture A is a co-culture comprising Penicillium janthinellum VUO 10,201 and bacterial consortium VUN 10,009. Co-culture B is a co-culture comprising Penicillium janthinellum VUO 10,201 and Stenotrophomonas maltophilia VUN 10,010.

FIG. 2 shows the degradation of chrysene and benz[a]anthracene in BSM by bacterial consortium VUN 10,009 (O), P. janthinellum VUO 10,201 (□) and co-culture A (▴). PAH-BSM media inoculated with a killed co-culture A (▾) is also shown. Culture media comprised BSM and a single PAH. Bacterial growth is shown as an estimated cell number determined in axenic cultures (O) and co-culture A (▴). Fungal dry weight is shown for axenic cultures (□) and co-culture A (Δ). Incubations were performed at 30° C./175 rpm in the dark.

FIG. 3 shows degradation of pyrene, benzo[a]pyrene and dibenz[a,h]anthracene in BSM by P. janthinellum VUO 10,201 (□), S. maltophilia VUN 10,010 (O) or co-culture B (▴). PAH-BSM media inoculated with a killed co-culture B (▾) is also shown. Culture medium comprised BSM and a single PAH. Bacterial growth is shown as an estimated cell number determined in axenic cultures (O) and co-culture B (●). Fungal dry weight is shown for axenic cultures (□) and co-culture B (▪). The incubations were performed at 25° C. in the dark.

FIG. 4 shows degradation of PAHs in PAH-spiked soil by bacterial consortium VUN 10,009, S. maltophilia VUN 10,010, P. janthinellum VUO 10,201, co-culture A or co-culture B. The soil contained three- to seven-benzene ring compounds. The amount of PAH remaining in the soil is shown for chrysene (O), benz[a]anthracene (□), benzo[a]pyrene (Δ), pyrene ( [symbol] ) and dibenz[a,h]anthracene ( ). The two panels for axenic P. janthinellum VUO 10,201 represent the same data. The incubations were performed at 30° C./175 rpm in the dark.

FIG. 5 shows mineralization of [¹⁴C]benzo[a]pyrene in BSM containing pyrene (250 mg l⁻¹) and benzo[a]pyrene (50 mg l⁻¹). ¹⁴CO₂ data are the cumulative amount recovered from medium incubated with either VUN 10,009 (●), S. maltophilia VUN 10,010 (▪), or the respective HgCl₂-killed cell controls (O,□). The incubations in BSM were performed at 30° C./175 rpm and soil culture incubations were performed at 25° C. All incubations were performed in the dark.

FIG. 6 shows [¹⁴C]benzo[a]pyrene mineralization in liquid and soil cultures. Labelled benzo[a]pyrene was added to BSM containing benzo[a]pyrene only (A and E), BSM containing benzo[a]pyrene and PAH mixture (B and F), PAH-spiked soil (C and G) and PAH-contaminated soil (D and H). The upper panels represent samples inoculated with axenic cultures of P. janthinellum VUO 10,201 (▴), bacterial consortium VUN 10,009 (▪) or co-culture A (♦). The lower panels represent samples inoculated with axenic cultures of P. janthinellum VUO 10,201 (Δ), S. maltophilia VUN 10,010 (□) or co-culture B ( ). Samples inoculated with HgCl₂-killed cells of either co-culture A (●) or co-culture B (O) are also shown. ¹⁴CO₂ evolution due to indigenous microbial activity in uninoculated PAH-contaminated soil (▾,∇) is also shown. The incubations were performed at 30° C./175 rpm in the dark.

FIG. 7 illustrates the production of unidentified compounds by P. janthinellum VUO 10,201 (A), bacterial consortium VUN 10,009 (B) and co-culture A (C) during incubation in BSM containing benzo[a]pyrene. The figure shows the peak areas of degradation products, as determined by HPLC of DCM extracts of cultures at day 28 of incubation. The incubations were performed at 30° C./175 rpm in the dark.

FIG. 8 shows the production of compounds formed during benzo[α]pyrene degradation by axenic cultures of P. janthinellum VUO 10,201, S. maltophilia VUN 10,010 and co-culture B over a 56 day period. Culture medium was BSM containing beno[α]pyrene as a sole source of carbon and energy. The data represent the peak areas of degradation compounds as determined by HPLC analysis.

FIG. 9 shows the production of compounds formed during benzo[a]pyrene degradation by axenic culture of P. janthinellum VUO 10,201, bacterial consortium VUN 10,009 and by co-culture A over a 56 day period. The culture medium was BSM containing benzo[a]pyrene as a sole source of carbon and energy. The data represent the peak areas of degradation products determined by HPLC analysis. The incubations were performed at 30° C./175 rpm in the dark.

FIG. 10 shows solublization of pyrene (A), fluoranthene (B) and benzo[a]pyrene (C) by Triton X-100 (□), Tergitol NP-10 (Δ), Brij 35 (O), Igepal CA-630 ( ) and Tyloxapol (∇). PAH solubility was measured in BSM at 30° C.

FIG. 11 shows pyrene degradation by S. maltophilia VUN 10,010. Pyrene was added to the medium either as pyrene crystals or presolubilized in DMF. The cultures contained the following: pyrene crystals in the presence (O) or absence (X) of Tergitol NP-10 (10 g l⁻¹); presolubilized pyrene in the presence (▴) or absence (▪) of Tergitol NP-10 (10 g l⁻¹). The incubations were performed in BSM at 30° C., using low initial cell populations.

FIG. 12 shows pyrene degradation by S. maltophilia VUN 10,010 in the presence of various surfactants. Apparent pyrene concentration (left hand panels) and cellular protein concentration (right hand panels) is shown for each culture. Each surfactant was added to the medium to achieve the following concentrations: 0.5 g l⁻¹ (▴), 5.0 g l⁻¹ (●) and 10.0 g l⁻¹ (▪). The control cultures ( ) contained no added surfactant. The incubations were performed in BSM at 30° C., using low initial cell populations.

FIG. 13 shows pyrene mineralization by S. maltophilia VUN 10,010 in the presence of 5.0 g l⁻¹ Tergitol NP-10 (▴), 5.0 g l⁻¹ Brij 35 (▪), or in the absence of surfactant (O). An inoculated control containing 0.2% HgCl₂ (X) is also shown. The incubations were performed in BSM at 30° C., using low initial cell populations.

FIG. 14 shows degradation of high molecular weight PAHs by high initial populations of S. maltophilia VUN 10,010 in the absence of surfactant (O) and in the presence of 5 (▴) or 10 (▪) g l⁻¹ Tergitol NP-10. Inoculated controls containing 0.2% HgCl₂ and 5 g l⁻¹ Tergitol NP-10 (X) are also shown. Incubations contained a single PAH in BSM at 30° C. The PAH remaining was calculated as a percentage relative to the PAH recovered from the inoculated control. Averages for the test cultures are from triplicate experiments.

FIG. 15 shows production of unidentified compounds by S. maltophilia VUN 10,010 during incubation in BSM containing benzo[a]pyrene and 10 g l⁻¹ Tergitol NP-10. The figures depicts HPLC profiles of DCM extract at time zero (A) and after 35 days incubation, showing the production of compounds I, II and III (B). Panel C shows the area of each peak identified by HPLC analysis of samples taken at regular intervals during the 49 day incubation. The incubations were conducted using high cell density cultures at 30° C.

FIG. 16 shows PAH degradation by S. maltophilia VUN 10,010 in a PAH mixture, with either 5 g l⁻¹ Tergitol NP-10 (closed symbols) or no added surfactant (open symbols). The incubations were conducted in BSM at 30° C., using low initial cell populations. The PAH remaining was calculated as a percentage relative to the PAH recovered from the uninoculated control. Averages for the test cultures are from triplicate experiments.

FIG. 17 shows the PAG concentrations in PAH-spiked soil containing a mixture of PAHs and Tergitol NP-10. PAHs represented are pyrene ( [symbol] ), chrysene (O), benz[a]anthracene (□), benzo[a]pyrene (Δ) and dibenz[a,h]anthracene ( ). Soils were amended with 2% (w/w) Tergitol NP-10 and high initial cell populations of axenic cultures of either bacteria (approximately 10⁶ cell g⁻¹ soil) or the fungus (25 mg[wet wt. of mycelia] per g soil), or the co-cultures comprising bacteria and the fungus were used to inoculate the soil. All incubations were performed at 25° C. in the dark.

### DETAILED DESCRIPTION OF THE INVENTION

The invention will now be described in detail by way of reference only to the following non-limiting examples and to the figures.

Materials and Methods

Chemicals

Fluorene and coronene were purchased from Aldrich Chem. Co. (Milwaukee, Wis.); phenanthrene, fluoranthene, pyrene, chrysene, benz[a]anthracene, benzo[a]pyrene and dibenz[a,h]anthracene, and surfactants were purchased from Sigma Chemical Company (St Louis, Mo.). All PAHs were high purity grade. [4,5,9,10-¹⁴C]Pyrene (58.7 mCi/mmol, radiochemical purity>98%) and [7-¹⁴C]benzo[a]pyrene (26.6 mCi/mmol, radiochemical purity>98%) were purchased from Sigma Chemical Company (St Louis, Mo.). Dichloromethane, N′,N′-dimethylformamide, methanol and other solvents and chemicals, except where specified, were obtained in analytical grade from BDH Laboratory Supplies (Poole, England).

Media

Bacteriological media, including nutrient agar, yeast extract agar (MEA) and agar were purchased from Oxoid (Unipath Ltd, Hampshire, England). Potato dextrose agar (PDA) and malt extract agar (MEA) were obtained from Difco Laboratories (Detroit, Mich.). The basal salts medium (BSM) contained per liter: 0.4 g K₂HPO₄, 0.4 g KH₂PO₄, 0.4 g (NH₄)₂SO₄, 0.3 g NaCl, 5 ml trace element solution, 5 ml vitamin solution and 5 ml magnesium/calcium solution (Juhasz et al, 1996). Vitamin, trace element and magnesium/calcium solutions were filter sterilized (0.22 μm membrane; Sartorius AG, Germany) and added to autoclaved BSM. Basal salt-glucose medium (BSMG) comprised BSM containing 1% glucose. MYPD broth consisted of 0.3% malt extract, 0.3% yeast extract, 0.5% peptone and 1% dextrose; the pH was adjusted to 6.0 (Launen et al, 1995).

Soils

The soil used for degradation and mineralization studies was an uncontaminated soil with no history of hydrocarbon contamination, and a petroleum-contaminated soil. The clean soil consisted of 93% sand, 11.6% clay and less than 0.5% silt.

The PAH-contaminated soil contained high levels of C10-C14 (350 ppm), C15-C28 (6,700 ppm) and C29-C36 (1,300 ppm) long chain hydrocarbons, lead (570 ppm), zinc (260 ppm) and the following PAHs: naphthalene (186 ppm), acenaphthalene (43 ppm), fluorene (87 ppm), phenanthrene (156 ppm), anthracene (53 ppm), fluoranthene (137 ppm), pyrene (99 ppm), benz[a]anthracene (33 ppm), benzo[a]pyrene (15 ppm) and dibenz[a,h]anthracene (12 ppm).

PAH-spiked soil was prepared by spiking uncontaminated soil with a stock solution of a PAH mixture to attain the final concentration of 100 mg kg⁻¹ soil for fluorene; 250 mg kg⁻¹ soil for phenanthrene and pyrene and 50 mg kg⁻¹ soil for fluoranthrene, benz[a]anthracene, chrysene, benzo[a]pyrene and dibenz[a,h]anthracene, and 25 mg kg⁻¹ soil for coronene.

Microorganisms

As described in detail below, the fungus Penicillium janthinellum VUN 10,201 and bacterial consortium VUN 10,009 were isolated from soil collected from a creosote-contaminated site in Warracknabeal, Victoria, Australia. The pseudomonad bacteria Stenotrophomonas maltophilia strains VUN 10,010 and VUN 10,012 and Pseudomonas fluorescens VUN 10,011 were isolated from contaminated soil from a manufactured gas plant, collected from a site in Port Melbourne, Victoria, Australia.

PAH Stock Solutions

Stock solutions of each PAH compound and PAH mixture were prepared in dimethylformamide (DMF). In single PAH degradation experiments, the stock solutions of individual PAHs were added to BSM to achieve final concentrations of 250 mg l⁻¹ for phenanthrene and pyrene 20 mg/ml⁻¹ for coronene, 50 mg l⁻¹ for fluoranthrene benz[a]anthracene, chrysene, benzo[a]pyrene and dibenz[a,h]anthracene, and 100 mg l⁻¹ for fluorene. In the experiments containing a PAH mixture, BSM was supplemented with a mixed-PAH stock solution to attain a final concentration of 100 mg l⁻¹ for fluorene, 250 mg l⁻¹ for phenanthrene and pyrene, 10 mg l⁻¹ for fluoranthrene, benz[a]anthracene, chrysene, benzo[a]pyrene and dibenz[a,h]anthracene, and 5 mg l⁻¹ for coronene, or 100 mg l⁻¹ for phenanthrene and pyrene; 20 mg l⁻¹ for fluorene; 10 mg l⁻¹ for fluoranthene, benz[a]anthracene, chrysene, benzo[a]pyrene and dibenz[a,h]anthracene, and 5 mg l⁻¹ for coronene.

Preparation of Bacterial and Fungal Inocula

Bacterial cells were grown in BSM (500 ml) supplemented with pyrene (250 mg l⁻¹); the incubation was performed in an Ehrlenmeyer flask (1 litre) at 30° C./175 rpm until growth reached late exponential phase. If a large number of cells were required, bacteria were cultured in a 15-litre fermenter using the same culture conditions. Bacteria were harvested by centrifugation, washed twice with sterile BSM and resuspended in an appropriate volume of BSM. These suspensions were used as inoculum. Killed bacterial cultures were prepared by using an inoculum of bacteria (approximately 10⁷ cell ml⁻¹) poisoned with HgCl₂ (0.7 g l⁻¹). Fungal inocula were prepared by growing P. janthinellum on PDA plates at 30° C. for 7 days. Sterile MYPD (25 ml) was then added to the plate with gentle agitation to suspend the spores. This suspension (10 ml) was used to inoculate sterile MYPD broth (250 ml). This broth was incubated for 48 hours at 30° C./175 rpm. At this stage, tiny mycelial pellets had formed and these were then filtered through Whatman no. 1 filter paper, and washed twice with sterile BSM. Killed fungal inocula were prepared by using an inoculum of 7 day-old MYPD cultures poisoned with HgCl₂ (0.7 g l⁻¹).

Biomass Determinations

Most probable number estimates for bacteria were conducted in 96-well microtitre plates. Culture samples were ten-fold serially diluted in 0.1% peptone water. For soil samples, 1 g of soil was suspended in Ringer's solution (9 ml) and mixed by vortexing. After precipitation of the solid soil particles, 1 ml of supernatant was removed for preparing a series of ten-fold dilutions. A volume (100 μl) from each dilution was inoculated into each of 3 or 5 replicate wells containing 100 μl of double-strength nutrient broth. All cultures were incubated at 30° C. for 2-7 days, and growth was visualised by the appearance of turbidity compared with controls (uninoculated medium and peptone water-inoculated medium). The number of positive wells in each set of three or five was tabulated, and the MPN of bacteria estimated from the appropriate MPN table (Collins et al, 1989). Dry weight was used to determine fungal biomass. Cultures were filtered using a Whatman No. 1 filter paper. The mycelia were washed with approximately 200 ml of deionised water and then dried at 105° C. until a constant weight was achieved. To allow for additional weight from organic and inorganic components in the medium, including possible bacterial biomass in the case of co-culture experiments, a sample of PAH medium without the fungus was similarly filtered, and its dry weight subtracted from the fungal dry weight measurements.

PAH Mineralization

PAH mineralization was measured by quantification of ¹⁴CO₂ in duplicate biometers. [¹⁴C]Pyrene or [¹⁴C]Benzo[a]pyrene (1 μl; specific radioactivity of 1 mCi ml⁻¹) and unlabelled pyrene or benzo[a]pyrene (50 mg l⁻¹) or PAH mixture was added to sterile BSM (100 ml) in a biometric flask (250 ml); the 50 ml side-arm contained 0.5 M NaOH (5 ml). The side arm was sealed with a rubber stopper pierced by a 15-gauge needle (15 cm long), which was used to withdraw samples for measuring ¹⁴CO₂ production. Cultures were then inoculated, and the biometers sealed with a rubber stopper containing a 0.22 μm filter (Sartorius AG, Germany). Pyrene-grown bacterial cells (ca. 10⁴ cells ml⁻¹) and 2 day-old mycelial pellets of P. janthinellum (25 mg wet weight ml⁻¹) were used as inoculum. Abiotic medium and killed-cell cultures served as controls. PAH mineralisation in liquid medium was conducted in the dark at 30° C./175 rpm. For [¹⁴C]benzo[a]pyrene mineralization in soils, 100 g of PAH-spiked soil or PAH-contaminated soil was mixed thoroughly with [¹⁴C]benzo[a]pyrene in the biometer flasks. This soil was inoculated with either the bacteria (10⁴ cells g⁻¹ soil), fungus (25 g wet weight kg⁻¹ soil) or co-cultures (10⁴ cells g⁻¹ soil, 25 g wet weight kg⁻¹ soil). Soil moisture was adjusted to approximately 65% of its water holding capacity. After inoculation, all reactors were incubated at 30° C. in the dark. The evolved ¹⁴CO₂ was trapped by the NaOH solution. At regular time intervals, the whole alkali content in the side-armed tube was taken for ¹⁴CO₂ analysis and the removed NaOH replaced with fresh alkali solution. Before taking the final samples for ¹⁴CO₂ measurement, a few drops of concentrated H₂SO₄ solution were added to the cultures and the acidified samples shaken for 5 minutes to release dissolved ¹⁴CO₂.

Mass Balance Analysis

Mass balance determination for [¹⁴C]pyrene or [¹⁴C]benzo[a]pyrene was quantified as percentage recovery of radioactivity in alkali solution, biomass and DCM-extractable fractions produced during mineralization of the radiolabelled PAH substrate. ¹⁴CO₂ was measured according to the procedure described for monitoring mineralisation of ¹⁴C-labelled compounds in aqueous samples (Fedorak et al, 1982). The removed NaOH (1 ml) was mixed with 9 ml of CytoScint™ scintillation cocktail (ICN, Research Product Division, Costa Mesa, Calif.) in 20 ml-scintillation vials (Crown Scientific Pty. Ltd., Australia), and the radioactivity was determined using a liquid scintillation counter (LSC; Wallac 1410, Pharmacia). Percentage conversion of radiolabeled PAH to bacterial biomass was determined by pelleting cells followed by resuspension in water. To remove PAHs adsorbed to the biomass, the resuspended bacterial cells were extracted with DCM. After separation of the two phases by centrifugation (4,000 rpm/5 min), the radioactivity in the organic extract was quantified by LSC. Then an aliquot of the extracted, resuspended cell solution was mixed with CytoScint™ scintillation cocktail and quantified by LSC. The cell-free supernatant obtained after the initial pelleting of bacterial cells was quantified by LSC to determine the percentage of PAH converted into aqueous soluble products. This aqueous phase was also extracted with DCM, and the organic soluble ¹⁴C-labelled products were quantified by LSC. Fungal cultures were treated slightly differently to the bacteria, since the fungal mycelium was sonicated for 30 sec over a 3 min sonication period using an ultrasonicator (Branson Sonifer 450). The sonicated material was then extracted with three 10 ml DCM portions; these three portions were subsequently pooled. Following DCM extraction, the mycelial debris was separated from the aqueous fraction by filtration through glass wool. CytoScint™ scintillation cocktail (9 ml) was then added to 1 ml portions of the DCM extract, the aqueous fractions and the entire mycelial fraction. Radioactivity was quantified by LSC. Mass balance analysis was not performed for soil samples, only ¹⁴CO₂ evolution was determined.

Mutagenicity Assays

The mutagenicity assays were conducted according to the revised methods of Maron and Ames (Maron and Ames, 1983), using Salmonella typhimurium tester strains TA98 and TA100. The bacterial mutagenicity assays were performed on the PAH compounds and crude extracts of metabolites formed during PAH degradation. All of the tested substances were dissolved in dimethyl sulfide (DMSO). To determine the direct mutagenicity activity, an aliquot of the tested substance (0.1 ml) was mixed with the tester strain culture (0.1 ml) (grown overnight and with a density of 10⁸ cells ml⁻¹) and 2.5 ml of molten top agar (50 μM L-histidine, 50 μM d-biotin, 0.5% NaCl, 0.6% agar) and poured onto minimal glucose plates (Vogel-Bonner medium E containing 2.0% glucose and 1.5% agar). Assays were also performed in the presence of hepatic post-mitochondrial supernatant (S9) prepared from male Sprague-Dawley rats pretreated with Aroclor 1254 (Moltox™ post-mitochondrial supernatant, Molecular Toxicology, Inc., Monsanto, USA). In the S9-mediated assays, the test substance (0.1 ml), the tester strain (0.1 ml), 0.5 ml of an S9 mix (0.2 ml of S9, 1.0 ml of MgCl₂-KCl salt, 0.25 ml of 1 M glucose-6-phosphate, 2.0 ml of 0.1 M NADP, 25 ml of 0.2 M sodium phosphate buffer, pH 7.4, adjusted to total volume of 50 ml) and molten top agar (2.0 ml) were mixed and poured onto plates containing Vogel's minimal glucose agar. Controls used in this test included plates with no compound addition, solvent control with DMSO added and positive control with known mutagen (aflatoxin) added. All assays were performed in triplicate. Revertant colonies were counted after 48 hours incubation at 37° C.

Gas Chromatography and High Performance Liquid Chromatography

PAH concentrations in the DCM extracts were measured on a Varian Star 3400 gas chromatograph, equipped with a flame ionization detector, using a BPX-5 capillary column (25 m×0.22 mm). The nitrogen carrier flow rate was 0.8 ml min⁻¹ with a pressure of 20 psi under split conditions (95:1). The oven temperature was programmed at 200° C. for 1 min, followed by a linear increase of 10° C. min⁻¹ to 320° C.; this temperature was held for 15 min. Injector and detector temperatures were maintained at 300° C. The peak areas of both internal standard and PAH were used to calculate peak area ratios. Ratios obtained for each sample, as well as controls, were compared to those of PAH standards. For the biodegradation experiments, the standard curves were linear in the concentration range of 0.5 to 250 mg l⁻¹ for pyrene and 0.5 to 50 mg l⁻¹ for chrysene, benz[a]anthracene, benzo[a]pyrene, and dibenz[a,h]anthracene.

Triplicate samples from biodegradation experiments were used for the extraction of PAH degradation products. The content from each culture vial (10 ml) was transferred to separating funnels (100 ml) and extracted twice with an equal volume of DCM. Cultures containing a fungal inoculum were sonicated as described above before extraction. The pH was adjusted to 2.5 with concentrated HCl and extracted twice again with an equal volume of DCM. The organic extracts were pooled and dried over anhydrous Na₂SO₄. The DCM phase was evaporated to partial dryness with a rotary evaporator and then dried completely using nitrogen gas. Dried samples were redissolved in methanol (200 μl) for HPLC analysis. Reverse-phase HPLC was performed on a Varian Star Liquid Chromatograph System comprising a 9012 solvent delivery system, a 9100 autosampler and a 9050 variable wavelength UV-VIS detector; this was controlled by Varian Star chromatographic software (Version 4.01). Dried organic extracts prepared from experimental samples, including abiotic and killed cell controls, were appropriately diluted and injected (70 μl) onto a Spherex 5 C₁₈ column (250 mm×4.6 mm id; Phenomenex, Torrance, Calif., USA). The solvent consisted of water and methanol, using the following gradient: 0 min, 50:50; 0 to 30 min, ramp to 0:100; 30 to 50 min, isocratic at 0:100. The flow rate was 0.7 ml min⁻¹. Compounds in the eluate were detected at 254 nm.

Various methods were tested for the dichloromethane extraction of PAHs and internal standard from the bacterial and fungal cultures in order to maximise the recovery of these compounds. For the bacterial cultures in BSM, greater than 99% of added PAHs was recovered from the first extraction by using a simple, but vigorous, shaking procedure. However, this technique was inappropriate for fungal cultures, since PAH recovery was adversely affected by the high adsorption of PAH compounds to the fungal mycelia. Three sequential extractions combining ultrasonication and vigorous shaking was required for the fungal cultures, and co-cultures, to recover approximately 97% to greater than 99% of the known PAH concentration. This sequential ultrasonication procedure was also suitable for extracting PAHs from spiked soil, in which the PAH recovery was relatively high (approximately 98% to greater than 99%). There was no significant difference in PAH extraction efficiency between this method and the standard Soxhlet extraction method. Results obtained from the sequential ultrasonication extraction method also showed good extraction reproducibility with no significant differences among replicate samples according to variance analysis at 95% confidence level.

### EXAMPLE 1

### Efficiency of PAH Extraction

The efficiency of the PAH extraction was evaluated by comparing the amount of PAH and internal standard (2,3-benzo[b]fluorene) recovered from liquid cultures or PAH-spiked soil to the known amount of these compounds which was added to the medium before extraction; these tests were performed in the presence and absence of killed-cell inocula. Sequential DCM extraction using three successive repeats was conducted, and the PAH concentrations measured after each extraction. Based on the data obtained, which showed high recovery and reproducibility, we employed a vigorous shaking procedure for PAH extraction from bacterial cultures in BSM, and three successive sequential ultrasonicated extractions for PAH extraction from fungal culture and soil.

Similar experiments were performed in the presence and absence of surfactant. Three successive sequential extractions were conducted, and the PAH concentration was measured after each extraction; vigorous shaking and sonication was used to improve mass transfer of PAHs (three to seven benzene rings) and internal standards into the DCM. In the absence of surfactant, greater than 99% of all the added PAH was recovered from the first extraction with no sonication being required. The extraction of PAHs from BSM was found to be slightly affected by the presence of surfactant (ca. 90% recovery on the first extraction). PAH extraction efficiency in the presence of surfactant was appreciably improved by the combination of ultrasonication and vigorous shaking, with the amount of PAH recovered from the first extraction being greater than 99%. In the light of these results, ultrasonication and vigorous shaking were adopted for all extractions performed in the following experiments.

After adding 2,3-benzo[b]fluorene (2,3-BF) as an internal standard (100 μl of a stock solution containing 1 mg ml⁻¹ in DCM) and DCM (1 ml), bacterial cultures were vigorously shaken for 20 sec and allowed to stand at room temperature 1-2 h before freezing overnight at −20□C. For fungal cultures, samples were placed on ice and sonicated (output control of seven and a duty cycle of 50%) for 30 seconds using a ⅛″ tapered microtip attached to a ½″ horn (Branson Sonifer 450); the total sonication time for each sample was 3 minutes (6×30 s). The extracts were separated from the mycelial debris by filtration through glass wool in a glass minicolumn. Mycelial extraction was repeated twice, and the extracts were filtered and pooled in a clean vial. All extracted samples were shaken vigorously and allowed to settle at room temperature for 1-2 hours, after which time two layers were visible; an upper water phase and the lower organic phase. This biphasic solution was frozen overnight at −20° C. After thawing, the vial was inverted, and approximately 500-800 μl of the heavier organic phase was withdrawn from the bottom using a 1 ml glass syringe (SGE, Australia). PAH in soil samples was extracted by sequential ultrasonication. Soil was mixed thoroughly with Na₂SO₃ before extraction. The same procedures as described for the extraction of PAHs from fungal culture were performed for soil samples. The three extracts were pooled, and PAH concentrations were determined by GC-FID.

### EXAMPLE 2

### Enrichment, Isolation and Identification of PAH-Degrading Microorganisms

PAH-contaminated soil samples were collected from an abandoned factory site located near Port Melbourne, Victoria, Australia. The site was previously used for a gas manufacturing plant operation, and until recently was used as a defence facility site. Enrichment and isolation of PAH-degrading bacteria was carried out using pyrene as a sole carbon and energy source. Firstly, a bacterial consortium was enriched by shaking 20 g (wet weight) of contaminated soil overnight in a 100 ml volume of Ringer's solution at 30° C. and 175 rpm; 5 ml of the supernatant was used to inoculate a 45 ml volume of BSM containing 100 mg l⁻¹ pyrene. When microbial growth was visible, a portion of culture broth was transferred to fresh pyrene-containing BSM at an inoculum size of 10% (v/v). After several successive subcultures, pure cultures were isolated as follows by the spray plate method (Kiyohara et al, 1982). Ten-fold serial dilutions of enriched culture in Ringer's solution were spread on BSM agar (BSM plus 1.5% bacteriological agar). The plates were then sprayed with a 2% ethereal solution of pyrene. After the ether had evaporated, plates were sealed with paraffin and incubated at 30° C. Pyrene degradation was detected by a distinct clear zone surrounding individual colonies. Single colonies were isolated, and selected isolates were stored at −20° C., in medium comprising 50% glycerol in BSM supplemented with pyrene (100 μg ml⁻¹), after being assigned a Victoria University culture collection number (VUN).

Gram staining and biochemical tests for bacterial identification were performed according to Collin et al. (1989) following growth in nutrient broth or nutrient agar. Isolates were tested for their ability to grow on various pyrene concentrations (250, 500 and 1,000 mg l⁻¹), as well as on different PAHs and 25 other carbon sources. Compounds tested were added to BSM as a sole carbon and energy source at 50 mg l⁻¹, and volatile compounds were supplied to the culture in the vapour phase. Starter cultures were grown in BSM-pyrene (250 mg l⁻¹) at 30° C. and 175 rpm for 7 days. The cells were collected and washed in BSM and the resuspended cells were inoculated into BSM containing different carbon sources. Inoculated BSM without an added carbon source and uninoculated BSM plus carbon source were used as controls. Inocula for the PAH degradation tests were transferred directly from parent cultures, but inocula for other carbon sources were washed prior to inoculation.

PAH-degrading bacteria able to use pyrene as a sole carbon source were isolated from contaminated soil using BSM containing pyrene (100 mg l⁻¹). Three distinct isolates, each of which was slightly different in colony shape and colour, were selected for storage. On the basis of biochemical tests, these three Gram-negative strains were tentatively identified as members of the genus Pseudomomas, and were designated strains VUN 10,010, VUN 10,011 and VUN 10,012.

These strains were further identified using 16S ribosomal RNA (rRNA) sequence analysis, using conventional methods. Approximately 80% of the 16S rRNA gene sequence of each strain was completed, and compared to DNA sequences in the GenBank database via the Australian National Genomic Information Service (ANGIS). Strains VUN 10,010 and VUN 10,012 showed a 90-100% sequence homology to S. maltophilia (formerly Pseudomonas maltophilia), and strain VUN 10,011 showed a 99-100% sequence homology to Pseudomonas fluorescens.

All the strains grew on the following compounds as the sole carbon and energy source, supplied at 50 mg l⁻¹: succinate, salicylic acid, pyruvate, benzoic acid, p-hydroxybenzoic acid, protocatechuic acid, Tween 20, Tween 40, Tween 60, Tween 80, catechol, phthalic acid, 4-chlorophenol, 2,5-dichlorophenol, benzene, toluene, n-hexadecane and n-octane. In addition, all three strains were able to degrade phenanthrene, fluorene and pyrene using unwashed pyrene-grown inocula (Table 1). The data for fluoranthene, chrysene, benz[a]anthracene and the five-benzene ring PAHs suggest that these compounds disappeared from the inoculated cultures at rates over and above those of the controls. However, as the differences in PAH concentration between the experimental and control cultures were small, degradation of the high molecular weight PAHs could not be confirmed from these data, although a slight increase in protein concentration was also observed in these cultures (data not shown). As strain VUN 10,010 appeared to show a slightly superior performance at reducing the concentration of high molecular weight PAHs, it was chosen for further PAH-degradation studies.

TABLE 1
PAH Degradation of PAH-Degradating Isolates

| | Incubation | % Degradation^{a} | | |
|---|---|---|---|---|
| PAH | Time (days) | VUN 10,010^{b} | VUN 10,011^{b} | VUN 10,012^{b} |
| Phenanthrene | 7 | 100.0 ± 0.0 | 100.0 ± 0.0 | 100.0 ± 0.0 |
| Fluorene | 7 | 100.0 ± 0.0 | 100.0 ± 0.0 | 100.0 ± 0.0 |
| Fluoranthene^{c} | 35 | 4.4 ± 2.4 | 2.1 ± 1.1 | 1.2 ± 1.3 |
| Pyrene | 10 | 100.0 ± 0.0 | 100.0 ± 0.0 | 96.7 ± 1.4 |
| Chrysene^{c} | 49 | 16.9 ± 6.5 | 16.5 ± 1.1 | 11.5 ± 0.6 |
| Benz[a]anthracene^{c} | 49 | 8.1 ± 3.2 | 7.1 ± 1.1 | 7.6 ± 1.1 |
| Dibenz[a,h]anthracene^{c} | 49 | 9.6 ± 2.4 | 12.5 ± 4.7 | 17.0 ± 4.1 |
| Benzo[a]pyrene^{c} | 49 | 12.2 ± 1.5 | 5.5 ± 2.0 | 8.6 ± 1.6 |
| Coronene^{c} | 49 | 3.9 ± 2.4 | 3.6 ± 1.3 | 2.3 ± 0.6 |

^{a}% degradation calculated relative to the amount of PAH removal from the uninoculated control; averages are from triplicate experiments.
^{b}Inocula were grown in BSM containing pyrene (250 mg I⁻¹) and unwashed cells transferred into BSM containing a single PAH as the sole carbon and energy source. Initial PAH concentrations were: phenanthrene (250 mg I⁻¹); fluorene, fluoranthene, pyrene and benz[a]anthracene (100 mg I⁻¹); chrysene, benzo[a]pyrene and dibenz[a,h]anthracene (50 mg I⁻¹); coronene (25 mg I⁻¹).
^{c}Only a slight increase in protein concentration was observed in these cultures.

### EXAMPLE 3

### Molecular Characterisation of Stenotrophomonas maltophilia Strain VUN10,010 and Comparison with Other S. maltrophilia Strains

Many of the strains described in Example 1 showed the capacity to degrade some PAHs and most had similar biochemical traits in that they:

a) were oxidase-positive, Gram negative, motile rods;

b) utilised glucose oxidatively;

c) cleaved procatechute via ortho cleavage; and

d) grew on succinic acid, maleic acid, pyruvate and several other organic substrates.

Strain VUN 10,011 produced a fluorescent pigment on fluorescein medium, and strain VUN 10,002 grew at 42° C., which distinguished these strains from other parallel isolates. Although the results of 20 biochemical tests did show some differential use of adonitol, rhamnose, raffinose, cinnamic acid, 4-chlorophenol, pentachlorophenol, toluene, nitrobenzene, gentisic acid and octane, many of the isolates showed similar traits and could not be readily differentiated.

Consequently, all of the stored VUN strains were further characterised to enable confirmation of their species status as S. maltophilia or other species, and differentiation between strains of S. maltophilia, particularly those which showed mineralisation of benzo a pyrene when cultured with Penicillium janthinellum.

16S rRNA genes were sequenced for each Gram negative isolate by isolating genomic DNA by obtaining polymerase chain reaction (PCR) amplification products, using a series of probes designed from the known sequence of the 16S rRNA gene of Burkholdena cepacia DSM 50181. The amplification products (approximately 450 bp to 1.5 bp, depending on the primer set used) were sequenced and the data analysed using the BLASTn program (Altschal et al., 1990). Internal sequences in these PCR products were identified and allowed new primer sets to be designed to generate further PCR products which overlapped in sequence with the original ones. The new PCR amplification products were also sequenced and the sequences aligned using the CLUSTAL W program (Higgins and Sharp, 1988). This enabled the entire 16S rRNA gene from each species to be determined. All sequences were registered with Genbank and the accession numbers for these are shown in Table 1.

The strains which had been tentatively assigned to the species S. maltophilia showed 98-99% sequence identity to the S. maltophilia type strain LMG958-T. It should be noted that the type strain does not degrade PAHs, and was isolated from a clinical source. This confirmed that the isolates were indeed S. maltophilia.

The pyrene-degrading strains of S. maltophilia were further typed by cleaving genomic DNA using six different restriction enzymes separately; the enzymes were selected because they did not cleave the 16S rRNA gene, and their cleavage sites were in six-base sequences: BamHI, EcoRI, BglII, HinDIII, EcoRV, and SalI. Enzymes which cleaved within the 16S rRNA gene, with six-base pair sites, were also used: SmaI, DraII, Eco0109I and Spel. The resulting digested genomic DNA fragments were separated using pulse-field gel electrophoresis (PFGE). The gels were viewed to determine the fingerprints arising from restriction enzyme digestion, and they were also probed using DNA fragments amplified from 16S rRNA genes to locate where the corresponding 16S rRNA genes were located in the digests. PCR-amplified rRNA genes were also digested with restriction enzymes to determine the pattern of digestion within these; SmaI and SpeI were particularly useful in differentiating the strains on the basis of these ribotype patterns. Phylogenetic analysis of the PFGE and ribotyping data showed that all of the S. maltophilia strains were distinct isolates, and that strain VUN 10,010, and other isolates, could be differentiated using these approaches.

TABLE 1
Strains and their Genbank accession no.

| Strain | Genus/species | Genbank accession no. |
|---|---|---|
| VUN10,001^{a} | S. maltophilia | AF100731 |
| VUN10,002^{a} | S. maltophilia | AF100732 |
| VUN10,003^{a} | S. maltophilia | AF100733 |
| VUN10,010^{a} | S. maltophilia | AF068008 |
| VUN10,012^{a} | S. maltophilia | AF068009 |
| VUN10,011^{a} | Pseudomonas | AF068010 |
| | fluorescens | |
| VUN10,013^{a} | Burkholderia cepacia | AF068011 |
| VUN10,035^{b} | S. maltophilia | AF100735 |
| VUN10,075^{b} | S. maltophilia | AF100734 |
| LMG958-T^{c} | S. maltophilia | X95923 |
| LMG11114^{c} | S. maltophilia | X95925 |

^{a}Isolated from a gas manufacturing plant
^{b}Isolated from uncontaminated water in rural Victoria
^{c}Type strains isolated from clinical sources

### EXAMPLE 4

### Enrichment, Isolation and Identification of Additional PAH-Degrading Microorganisms

In a second experiment, enrichment of PAH-degrading microorganisms was performed using benzo[a]pyrene (50 mg l⁻¹) as a sole source of carbon and energy. Firstly, a microbial consortium was enriched by shaking 20 g (wet weight) of contaminated soil overnight in Ringer's solution (100 ml) at 30° C. and 175 rpm; 5 ml of the supernatant was used to inoculate BSM (45 ml) containing 50 mg l⁻¹ benzo[a]pyrene. When microbial growth was visible, a portion of culture broth was inoculated (10% v/v) to fresh benzo[a]pyrene-containing BSM. Microorganisms were then isolated from this enriched culture after several successive transfers.

Bacteria alone could not be isolated on benzo[α]pyrene, and therefore bacterial isolation was performed using pyrene as a sole carbon source.

To isolate bacteria, fresh BSM containing pyrene (100 mg l⁻¹) and supplemented with cycloheximide (0.1 g l⁻¹) was inoculated with the enriched culture (0.1% v/v). To ensure that no fungi were present in this culture, five portions of the culture (0.1 ml each) were spread on PDA plates after every transfer. To isolate the fungi, the benzo[a]pyrene-enriched culture was diluted 10-fold into portions and spread over PDA plates supplemented with filter-sterilised penicillin G (60 mg ml⁻¹), streptomycin sulphate (100 μg ml⁻¹) and benzo[a]pyrene (50 μg ml⁻¹). All plates were incubated at room temperature. After various incubation periods (up to several weeks), fungal colonies were selected and serially re-plated on the same medium without antibiotics until pure colonies were obtained. At this stage, all single fungal colonies had similar macroscopic characteristics; a pure single colony was then selected for storage. Stock fungal cultures were maintained on slants of PDA supplemented with benzo[a]pyrene (50 mg l⁻¹) and stored at 4° C.; these were sub-cultured every two months. Bacterial cultures were maintained at −20° C. in BSM-glycerol medium containing pyrene (100 mg l⁻¹). Freeze-dried stock cultures of bacteria and the fungus were also prepared, and maintained at −70° C.

A mixed microbial population was obtained from creosote-contaminated soil enriched with benzo[a]pyrene as a sole source of carbon and energy. Each successive transfer of the enrichment culture was found to contain both bacteria and fungi. A pure fungal isolate, identified as Penicillium janthinellum on the basis of macroscopic and microscopic characteristics, was obtained from this enriched culture. This fungus was assigned the Victoria University of Technology culture collection number of VUN 10,201. By inhibiting fungal growth, a consortium of bacteria was isolated on pyrene as sole carbon and energy source. This bacterial population was designated as VUN 10,009.

A pyrene-degrading pure culture was isolated from bacterial consortium VUN 10,009, and this was assigned the Victorian University culture collection number of VUN 10,013. This strain was identified using 16S ribosomal RNA (rRNA) sequence analysis, and was found to have a greater than 97% homology to Burkholderia sp.

### EXAMPLE 5

### Degradation of High Molecular Weight PAHs by Bacterial and Fungal Isolates

PAH degradation experiments using axenic bacterial and fungal cultures, and fungal-bacterial co-culture, were performed in 30 ml reaction vials containing 10 ml of medium. Before inoculation, 0.1 ml of PAH stock solution (either single PAH or mixed PAH stock as described above) was introduced into the medium. This medium was then inoculated with microorganisms to produce either axenic cultures or co-cultures. Axenic cultures were inoculated with either bacteria (1 ml) to provide an initial cell population of approximately 10⁴ cells ml⁻¹, or with mycelial pellets (0.25 g wet weight) in 10 ml of BSM. For the co-culture experiments, BSM (10 ml) contained bacteria (10⁴ cells ml⁻¹) and 0.25 g wet weight of mycelial pellets. Experiments to investigate of co-metabolic PAH degradation by P. janthinellum were conducted with the PAH in BSM supplemented with glucose (1% final concentration) or MYPD medium. Co-metabolic studies with bacteria were performed with the PAH in BSM supplemented with pyrene (250 mg l⁻¹). Medium (10 ml) was inoculated with fungal or bacterial culture using the same inoculum size as described above. All cultures were aseptically flushed with filtered (0.22 μm) air (0.2 litres min⁻¹) for 5 seconds on the day of inoculation and every 7 days thereafter. Abiotic controls were sterile medium containing only PAH(s). Killed-cell controls were the PAH-containing medium inoculated with a killed microbial population. The experimental cultures were conducted in triplicate, and all controls were run in duplicate. All cultures were incubated in the dark at 30° C./175 rpm. At designated time intervals the entire culture (10 ml) was sacrificed for the PAH-extraction procedure and biomass determinations.

Axenic cultures of S. maltophilia VUN 10,010 and bacterial consortium VUN 10,009 were able to degrade PAHs containing up to five benzene rings as the sole carbon source in BSM, as shown in Table 2. However, the degradation of chrysene, benz[a]anthracene, benzo[a]pyrene and dibenz[a,h]anthracene was very slow, with 6-12% of these compounds being removed over 56 days, and there was no growth of either VUN 10,010 or of the bacterial consortium under these conditions.

TABLE 2
Degradation of High Molecular Weight PAHs by PAH-Degrading Microorganisms

| | PAH removal (%)^{a} | | | | | | |
|---|---|---|---|---|---|---|---|
| | Bacterial consortium^{c} | | VUN 10,010^{c} | | P. janthinellum^{d} | | |
| PAH^{b} | BSM^{e} | BSM + PYRƒ | BSM^{e} | BSM + PYRƒ | BSM^{e} | BSM^{g} | MYPD^{g} |
| Pyrene | 100.0 + 0.0 | NT | 100.0 + 0.0 | NT | 3.3 + 0.3 | 80.3 + 2.8 | 93.6 + 2.8 |
| Chrysene | 8.2 + 0.5 | 12.1 + 0.4 | 9.1 + 1.2 | 28.3 + 1.2 | 12.2 + 0.4 | 25.6 + 1.9 | 31.7 + 3.7 |
| Benz[a]anthracene | 7.8 + 0.3 | 9.6 + 0.7 | 8.2 + 1.6 | 19.84 + 0.3 | 9.1 + 1.4 | 18.1 + 2.3 | 23.1 + 1.2 |
| Benzo[a]pyrene | 6.7 + 0.9 | 23.3 + 0.5 | 12.3 + 1.0 | 32.2 + 1.2 | 16.9 + 0.2 | 56.5 + 2.4 | 72.2 + 3.2 |
| Dibenz[a, h]anthracene | 5.9 + 0.5 | 13.8 + 0.9 | 7.8 + 0.6 | 26.7 + 0.7 | 12.9 + 0.3 | 24.4 + 2.7 | 36.7 + 1.7 |

^{a}% degradation calculated relative to the amount of PAH removed from abiotic and killed cell controls; averages are from triplicate experiments.
NT, not tested.
^{b}Initial PAH concentrations were 250 mg l⁻¹ for pyrene and 50 mg l⁻¹ for chrysene, benz[a]anthracene, benzo[a]pyrene, and dibenz[a, h]anthracene.
^{c}Inocula were grown in BSM containing pyrene (250 mg l⁻¹). Initial bacterial population was approximately 104 cells ml⁻¹.
^{d}Inocula consisted of 0.25 g of 2-day old mycelia grown in MYPD. This was inoculated into 10 ml of medium.
^{e}Data represent PAH concentrations in 56 day samples.
^{f}Pyrene (PYR; 250 mg l⁻¹) was supplied to BSM as a carbon substrate. Data represents PAH concentrations in 28 day samples.
^{g}Data represent PAH concentrations in 28 day samples

For a majority of the results shown in Table 2, PAH disappearance from microbial cultures was described as degradation, since the amount removed was in addition to the amount that disappeared from the corresponding killed-cell control. However, the extent of PAH biotransformation cannot be deduced from these data. There was a considerable improvement in the degradation of chrysene, benz[a]anthracene, benzo[a]pyrene and dibenz[a,h]anthracene by pure cultures of either VUN 10,009 or VUN 10,010 in BSM when pyrene (250 mg l⁻¹) was added to the culture. Compared to the bacterial consortium, VUN 10,010 degraded twice as much chrysene, benz[a]anthracene and dibenz[a,h]anthracene, and 1.4 times more benzo[a]pyrene under these co-metabolic conditions. Axenic cultures of P. janthinellum VUO 10,201 degraded substantial amounts of the four- and five-ring PAHs in nutrient-rich medium or BSM supplemented with glucose (1%), but these PAHs were slowly degraded and failed to support growth when supplied as a sole carbon source in BSM; it is questionable as to whether VUO 10,201 can degrade pyrene as a sole carbon source.

### EXAMPLE 6

### PAH Degradation by Fungal-Bacterial Co-Cultures

The inability of axenic cultures of bacterial consortium VUN 10,009 and P. janthinellum VUN 10,201 to grow on benzo[a]pyrene as a sole carbon and energy source was unexpected, since these organisms were enriched from PAH-contaminated soil using this compound. However, during enrichment it was observed that bacterial growth always occurred alongside fungal growth, suggesting that these organisms may need to be incubated together for growth on benzo[a]pyrene to occur. An experiment was therefore conducted to investigate the growth of bacterial consortium VUN 10,009 and P. janthinellum VUO 10,201 strains when combined as a co-culture (designated co-culture A) in BSM containing a single high molecular weight PAH as only source of carbon and energy. As shown in FIG. 1, P. janthinellum and the bacterial consortium both grew on benzo[a]pyrene and dibenz[a,h]anthracene as a sole carbon and energy source when incubated as a co-culture; no significant growth on these PAHs was observed in axenic cultures of these organisms. The bacterial population increased by at least two logs, from 10⁴ to 10⁶ cells ml⁻¹, and fungal dry weight increased by around 50-70% over a 56 day period.

Co-culture A also degraded a significantly greater amount of benzo[a]pyrene (26%) and dibenz[a,h]anthracene (19%) compared to axenic cultures of bacterial consortium VUN 10,009 (9.7% benzo[a]pyrene, 9.2% dibenz[a,h]anthracene) and P. janthinellum VUO 10,201 (17% benzo[a]pyrene, 13% dibenz[a,h]anthracene). Similar results were obtained for chrysene and benz[a]anthracene, as shown in FIG. 3. Pyrene degradation was similarly affected by co-culture A and by the axenic bacterial culture, with the only difference being that P. janthinellum was able to grow in BSM containing pyrene when the bacterial consortium was also present.

PAH degradation by fungal-bacterial co-cultures was further examined by combining P. janthinellum VUO 10,201 with Stenotrophomonas maltophilia VUN 10,010, a PAH-degrading bacterium isolated from a different site to VUO 10,201 (designated co-culture B). Preliminary work using dual cultures on PDA and NA plates showed that there was no apparent antagonistic effect between VUO 10,201 and VUN 10,010. Co-culture B was inoculated into BSM containing a single high molecular weight PAH as the sole carbon and energy source. As shown in FIG. 3, P. janthinellum VUO 10,201 and S. maltophilia VUN 10,010 both grew on benzo[a]pyrene or dibenz[a,h]anthracene when these two organisms were incubated together; growth on these PAHs did not occur in axenic cultures of these organisms. The rate of five-benzene ring PAH degradation by co-culture B was substantially higher than that observed in the axenic cultures and in co-culture A.

After 56 days, co-culture B degraded 59% (29.5 mg l⁻¹) benzo[a]pyrene and 35% (17.5 mg l⁻¹) dibenz[a,h]anthracene, compared to 27% (13.5 mg l⁻¹) benzo[a]pyrene and 19% (9.5 mg l⁻¹) dibenz[a,h]anthracene by co-culture A over the same time period (FIGS. 1 to 3). In BSM containing pyrene, the pyrene degradation rate and fungal biomass yield was greater using co-culture B (ca. 41.7 mg l⁻¹ day⁻¹, 3.6 mg ml⁻¹ dry weight) than using co-culture A (ca. 18.3 mg l⁻¹ day⁻¹, 3.1 mg ml⁻¹ dry weight). The improved pyrene degradation rate was probably due to the superior pyrene-degrading ability of VUN 10,010 compared to the bacterial consortium, rather than to the co-culture per se.

### EXAMPLE 7

### PAH Degradation in PAH-Spiked Soil by Fungal-Bacterial Co-Cultures

PAH degradation by co-cultures and axenic cultures of strains VUO 10,201, VUN 10,010 and the bacterial consortium was tested in uncontaminated soil spiked with a PAH mixture consisting of three- to seven-benzene ring compounds (designated PAH-spiked soil).

Sterile clean soil was artificially contaminated by adding a defined PAH mixture, prepared in DCM, to a sterile jar (1.5 litre capacity), allowing the solvent to evaporate and then adding clean soil to the jar. The soil was well mixed to form a homogenous mixture. The homogeneity of the mixture was confirmed before commencing experiments by testing the PAH concentration of five random portions of the soil. The relative standard deviation obtained between the PAH concentrations in five samples was lower than 1.5%. This PAH-spiked soil was distributed to sterile 1.5 litre jars, each jar receiving amounts equal to 200 g dry weight of soil. Bacteria were inoculated into this soil to give an initial bacterial population of 10⁷ cells g⁻¹ soil. In case of fungal-inoculated soil, mycelia were applied to attain the initial biomass of 25 g kg⁻¹ soil. The same bacterial and fungal inoculum size was used for axenic soil cultures and for co-culture of inoculated soil. Killed-cell controls were soils inoculated with a corresponding killed inoculum. Abiotic controls were uninoculated soil receiving a PAH mixture. Inoculated and uninoculated soil samples were supplemented with sterile BSM solution to approximately 65% of the soil water holding capacity. All treatments and controls were incubated at ambient temperature in the dark. At designated time intervals, triplicate samples of 1 g of soil from each jar were collected for analysis of PAH concentration.

The fate of pyrene, chrysene, benz[a]anthracene, benzo[a]pyrene and dibenz[a,h]anthracene in the PAH-spiked soil was monitored over 100 days. The results are shown in FIG. 4. Pyrene was rapidly degraded in soil inoculated with either S. maltophilia VUN 10,010 or the bacterial consortium, with all the pyrene (250 mg kg⁻¹ soil) being degraded in around 16-18 days. The degradation rate of chrysene, benz[a]anthracene, benzo[a]pyrene and dibenz[a,h]anthracene was slow in soil inoculated with only the bacterial consortium or with VUN 10,010. The amount of these PAHs degraded over 100 days was in the range of 16-32% for VUN 10,010 and 12-22% for the bacterial consortium, and long degradation lag periods of around 30 days (VUN 10,010) and 40-60 days (bacterial consortium) were observed. On the other hand, there was no detectable degradation lag period in soils inoculated with P. janthinellum VUO 10,201, and the PAH degradation rate was relatively high in the first 30-40 days compared to soils inoculated with only the bacteria. The amount of chrysene, benz[a]anthracene, benzo[a]pyrene and dibenz[a,h]anthracene degraded by VUO 10,201 after 100 days was in the range 24-48%; however, the degradation of each PAH all but ceased after 60 days. Although we did not determine fungal growth in the soil cultures, viable fungal biomass was still present in the soil after 100 days.

There was a substantial improvement in PAH degradation when the PAH-spiked soil was inoculated with either co-culture A or co-culture B. The amount of chrysene, benz[a]anthracene, benzo[a]pyrene and dibenz[a,h]anthracene degraded after 100 days was in the range 40-68% (co-culture A) and 44-80% (co-culture B). Furthermore, soils inoculated with the co-cultures did not show any apparent degradation lag period, and PAH degradation occurred during the entire incubation period. Bacterial populations showed similar growth profiles, with bacterial populations increased 100-fold, in soils inoculated with either axenic bacterial inocula or with co-cultures A and B.

### EXAMPLE 8

### Benzo[a]pyrene Mineralization by Fungal Bacterial Co-Cultures

S. maltophilia VUN 10,010 was shown in Example 8 to mineralise pyrene as a sole carbon and energy source, and bacterial consortium VUN 10,009 was also able to mineralise pyrene as a sole carbon source in BSM; 56% of added ¹⁴C was recovered as ¹⁴CO₂ in 20 days. Benzo[a]pyrene mineralisation by VUN 10,010 and VUN 10,009 was investigated under cometabolic conditions by adding [¹⁴C]benzo[a]pyrene to BSM containing pyrene (250 mg l⁻¹) and benzo[a]pyrene (50 mg l⁻¹). From FIG. 5, it can be seen that axenic cultures of VUN 10,010 and VUN 10,009 co-metabolically mineralised benzo[a]pyrene in the presence of pyrene. The rate of benzo[a]pyrene mineralisation by VUN 10,010 was substantially higher than that with VUN 10,009, with ¹⁴CO₂ evolution after 56 days being 32.8% and 13.0% respectively. The remaining labelled carbon was found mainly in the DCM extract, most likely in the form of undegraded benzo[a]pyrene and non-polar degradation products. These results are summarized in Table 3. No ¹⁴CO₂ was liberated from [¹⁴C]benzo[a]pyrene when this was added to axenic P. janthinellum VUN 10,201 cultures comprising benzo[a]pyrene and pyrene in BSM, benzo[a]pyrene in MYPD or benzo[a]pyrene and glucose in BSM. A high proportion of the ¹⁴C was recovered in the aqueous phase, probably as polar products generated from the partial degradation of benzo[a]pyrene.

TABLE 3
Distribution of ¹⁴C-residues After Incubation of
PAH-Degrading Organisms on [¹⁴C]Benzo[a]Pyrene

| | | | | | |
|---|---|---|---|---|---|
| | | | Aqueous | DCM | |
| Microorganism | ¹⁴CO₂ | Biomass | phase | phase | % Recovery |

BSM containing benzo[a]pyrene (50 mg l⁻¹)

| | | | | | |
|---|---|---|---|---|---|
| VUN 10,009 | NA | NA | 1.2 | 99.1 | 100.3 |
| VUN 10,010 | NA | NA | 1.6 | 98.5 | 100.1 |
| VUO 10,201 | NA | NA | 13.7 | 85.4 | 99.1 |
| Co-culture A | 16.3 | 7.2 | 2.2 | 73.4 | 99.1 |
| Co-culture B | 25.5 | 9.3 | 1.7 | 62.9 | 99.4 |

BSM containing benzo[a]pyrene (50 mg l⁻¹) and pyrene (250 mg l⁻¹)

| | | | | | |
|---|---|---|---|---|---|
| VUN 10,009 | 12.8 | 0.7 | 2.3 | 84.3 | 100.1 |
| VUN 10,010 | 32.4 | NA | 2.7 | 64.8 | 99.9 |
| VUO 10,201 | NA | NA | 14.5 | 84.1 | 98.6 |
| Co-culture A | 37.2 | 7.3 | 1.1 | 53.7 | 99.3 |
| Co-culture B | 58.1 | 8.1 | NA | 30.9 | 97.1 |

BSM containing a PAH mixture

| | | | | | |
|---|---|---|---|---|---|
| VUN 10,009 | 8.2 | 1.2 | 2.1 | 87.7 | 99.2 |
| VUN 10,010 | 32.8 | 1.7 | 2.3 | 62.6 | 99.4 |
| VUO 10,201 | NA | NA | 13.2 | 85.7 | 98.9 |
| Co-culture A | 35.7 | 6.9 | 1.4 | 55.3 | 99.3 |
| Co-culture B | 53.0 | 8.5 | 0.6 | 35.8 | 97.9 |

PAH-spiked soil

| | | | | | |
|---|---|---|---|---|---|
| VUN 10,009 | 12.3 | ND | ND | 84.8 | 97.1 |
| VUN 10,010 | 24.1 | ND | ND | 73.9 | 98.0 |
| VUO 10,201 | NA | ND | ND | 97.7 | 97.7 |
| Co-culture A | 37.7 | ND | ND | 61.4 | 99.1 |
| Co-culture B | 44.7 | ND | ND | 53.6 | 98.3 |

PAH-contaminated soil

| | | | | | |
|---|---|---|---|---|---|
| Indigenous | 4.8 | ND | ND | 93.1 | 97.9 |
| VUN 10,009 | 18.8 | ND | ND | 80.6 | 99.4 |
| VUN 10,010 | 31.5 | ND | ND | 67.3 | 98.8 |
| VUO 10,201 | 8.7 | ND | ND | 89.1 | 97.8 |
| Co-culture A | 37.7 | ND | ND | 60.8 | 98.5 |
| Co-culture B | 53.2 | ND | ND | 44.2 | 97.4 |

NA, Not appreciable.

ND, Not determined.

Co-cultures A and B mineralised benzo[a]pyrene as a sole carbon and energy source in BSM. The amount of ¹⁴CO₂ that evolved from [¹⁴C]benzo[a]pyrene was 16.3% after 56 days using co-culture A, and 25.5% using co-culture B, respectively, as shown in FIGS. 6A and 6B. No significant ¹⁴CO₂ evolution was detected from axenic cultures or the killed-cell controls. A ¹⁴C balance analysis at the end of the experiment identified less than 3% of radiolabeled carbon in the aqueous phase, with the majority (approximately 63-74%) being recovered in the DCM extract (Table 3). A relatively high amount of ¹⁴C was recovered in the co-culture biomass, indicating its incorporation into cellular material.

Co-culture A and co-culture B also mineralised substantial amounts of [¹⁴C]benzo[a]pyrene in BSM containing a PAH mixture (three- to five-benzene rings), with the amount of initial ¹⁴C recovered as ¹⁴CO₂ being 36% and 53%, respectively, after 56 days, as shown in FIGS. 6B and 6F); approximately 8% and 33% of ¹⁴C was recovered as ¹⁴CO₂ from the respective axenic bacterial cultures over 56 days. In agreement with the single PAH experiments, the remaining ¹⁴C in the co-cultures was found mostly in the DCM extract and the biomass (Table 3).

Benzo[a]pyrene mineralisation by co-cultures A and B was further tested in PAH-spiked soil and in PAH-contaminated soil obtained from a petroleum-polluted site (designated PAH-contaminated soil). For the PAH-spiked soil, no ¹⁴CO₂ evolved during the first 14 days of incubation, but over the next 86 days 38% and 45% of the initial radioactivity was recovered as ¹⁴CO₂ from co-cultures A and B, respectively. This is shown in FIGS. 6C and 6G. Benzo[a]pyrene mineralisation by the co-culture was superior to that observed in soil inoculated with axenic bacterial inocula, in which the amount of ¹⁴CO₂ evolved was approximately 12% with VUN 10,009 and 24% with VUN 10,010; no ¹⁴CO₂ evolution was recorded for soils inoculated only with P. janthinellum VUO 10,201 or for uninoculated soils. Similar results were observed in PAH-contaminated soil, benzo[a]pyrene mineralization by the co-culture apparently being unaffected by the adverse soil environment, as shown in FIGS. 6D and 6H. In fact, co-culture B mineralised benzo[a]pyrene to a greater extent (53% of initial ¹⁴C recovered as ¹⁴CO₂ after 100 days), and without an apparent lag period, compared to its performance in the PAH-spiked soil. This may be due to the indigenous microflora cooperatively mineralising benzo[a]pyrene with the co-cultures. Evidence of this is seen with the soil inoculated with axenic P. janthinellum VUO 10,201, in which 8.7% of the initial radioactivity is recovered as ¹⁴CO₂; this did not occur in PAH-spiked soil which did not contain a measurable indigenous microbial population. The indigenous soil microbial population mineralised only 5% of the added [¹⁴C]benzo[a]pyrene to ¹⁴CO₂ over 100 days. The balance of ¹⁴C added to the soils which was not recovered as ¹⁴CO₂ was found in the DCM extract (Table 3).

### EXAMPLE 9

### HPLC Analysis of PAH Degradation Products

DCM extracts from co-cultures and from axenic bacterial and fungal cultures comprising BSM with benzo[a]pyrene as a sole carbon and energy source were analysed for PAH degradation products by HPLC. One degradation compound, designated compound I, was observed to accumulate in axenic P. janthinellum VUO 10,201 cultures over the 56 day incubation, as illustrated in FIGS. 7 and 8. In co-culture A, compound I was the first peak to appear but, in contrast to axenic P. janthinellum VUO 10,201 cultures, the concentration of this compound decreased in the later stages of the incubation. Compound I was not present in axenic cultures of bacterial consortium VUN 10,009; however, three additional degradation compounds, designated compounds II, III and IV, were detected. Compound II was the first to appear in axenic bacterial consortium cultures, and its concentration initially increased, but then decreased to a constant level during the remainder of the 56 day incubation. Compounds II and IV first appeared in the 28 day sample, and their concentrations initially increased but then were constant during the remainder of the 56 day incubation. Compounds II, III and IV were also present in the 14 and 28 day samples of co-culture A, but these compounds were apparently degraded to other products by the co-culture, since they were not detected in the 56 day sample. A degradation compound, designated compound V, appeared in the co-culture but was not detected in the axenic cultures. The concentration of compound V increased up to 28 days, then decreased, and compound V was not detected in the 56 day sample. Similar results were observed with axenic VUN 10,010 cultures and co-culture B, as shown in FIG. 8.

### EXAMPLE 10

### Reduction in Mutagenicity of DCM Extracts

The microbial degradation of PAHs can result in either a reduction or an increase in the mutagenic potential of a PAH-contaminated medium, depending on the mutagenicity of the degradation products. The mutagenic potential of DCM extracts from a culture medium containing a particular PAH was assessed following incubation with either a co-culture or axenic bacterial and fungal cultures. As shown in Table 4, most of the axenic cultures failed to significantly reduce the mutagenic potential of BSM containing either benzo[a]pyrene or dibenz[a,h]-anthracene as the sole carbon and energy source.

TABLE 4
Mutagenicity of PAH extracts from liquid and soil cultures.

| | Number of revertants per plate⁸ | | | | | | | | | | | |
|---|---|---|---|---|---|---|---|---|---|---|---|---|
| | Benzo[a]pyrene^{b} | | | Dibenz[a, h]anthracene^{b} | | | PAH-spiked soil | | | PAH-contaminated soil | | |
| Extracts | Day 0 | Day 28 | Day 56 | Day 0 | Day 28 | Day 56 | Day 0 | Day 56 | Day 100 | Day 0 | Day 56 | Day 100 |
| Unin- | 687 ± 6 | 704 ± 8 | 701 ± 6 | 444 ± 5 | 438 ± 6 | 447 ± 7 | 689 ± 11 | 671 ± 14 | 693 ± 6 | 718 ± 4 | 711 ± 8 | 689 ± 4 |
| oculated | | | | | | | | | | | | |
| control^{b} | | | | | | | | | | | | |
| VUN | 696 ± 8 | 688 ± 9 | 657 ± 6 | 437 ± 7 | 411 ± 6 | 388 ± 1 | 702 ± 14 | 686 ± 9 | 594 ± 7 | 724 ± 9 | 703 ± 11 | 677 ± 12 |
| 10,009 | | | | | | | | | | | | |
| VUN | 682 ± 3 | 651 ± 8 | 632 ± 13 | 441 ± 4 | 407 ± 5 | 384 ± 2 | 695 ± 7 | 623 ± 10 | 501 ± 16 | 722 ± 20 | 697 ± 6 | 648 ± 4 |
| 10,010 | | | | | | | | | | | | |
| VUO | 673 ± 10 | 337 ± 13 | 339 ± 4 | 452 ± 7 | 392 ± 3 | 382 ± 8 | 697 ± 5 | 477 ± 12 | 367 ± 8 | 716 ± 7 | 634 ± 3 | 461 ± 8 |
| 10,201 | | | | | | | | | | | | |
| Co- | 682 ± 6 | 306 ± 11 | 272 ± 7 | 449 ± 10 | 296 ± 8 | 291 ± 4 | 705 ± 3 | 392 ± 10 | 295 ± 11 | 722 ± 8 | 617 ± 13 | 420 ± 6 |
| culture | | | | | | | | | | | | |
| A | | | | | | | | | | | | |
| Co- | 687 ± 14 | 294 ± 5 | 253 ± 9 | 460 ± 16 | 277 ± 10 | 276 ± 3 | 698 ± 7 | 367 ± 15 | 263 ± 6 | 719 ± 4 | 576 ± 6 | 409 ± 4 |
| culture | | | | | | | | | | | | |
| B | | | | | | | | | | | | |

^{a}Assay was performed using Salmonella typhimurium TA100 with the post-mitochondrial activation system (S9). DMSO served as solvent control for spontaneous mutation, for which the number of revertants per plate was 124 ± 3.
^{b}DCM extracts from cultures comprising BSM and either benzo[a]pyrene or dibenz[a, h]anthracene.

One exception was the axenic P. janthinellum VUN 10,201 culture, which reduced the mutagenicity of benzo[a]pyrene in BSM by around 50%. Both co-culture A and co-culture B significantly reduced the mutagenicity of the BSM containing benzo[a]pyrene (ca 60-63%) or dibenz[a,h]anthracene (ca 35-40%); these results were similar for both co-cultures, although co-culture B always reduced the mutagenicity to a slightly higher extent than co-culture A. The mutagenic potential of DCM extracts from PAH-spiked soil and PAH-contaminated soil was not significantly reduced over 100 days following inoculation with axenic bacterial inocula. On the other hand, amendment of these soils with P. janthinellum inocula resulted in a reduction in the mutagenicity of the DCM extracts of 47% for PAH-spiked soil, and 35% for PAH-contaminated soil). The mutagenic potential of DCM extracts from these soils was reduced even further by the co-cultures, the reduction being 58-62% for PAH-spiked soil and 42-43% for PAH-contaminated soil; the difference in the reduction of mutagenicity by co-culture A and co-culture B was not significant.

### EXAMPLE 11

### Microbial Growth in the Presence of Surfactants

Several criteria have been postulated regarding the use of solublizing agents as a means to improve PAH biodegradation, the most important being that the surfactant must not be toxic to the PAH-degraders and should not serve as a preferred substrate (Mulkins-Phillips and Stewart, 1974)

Bacterial growth on surfactants was tested in duplicate in BSM containing each surfactant as a sole carbon source, using starter cultures prepared as in Example 2. Sterilized BSM containing an individual surfactant (0.1 g l⁻¹) was prepared in 30 ml-reaction vials and inoculated with pyrene-grown cells. Growth was monitored by observing changes in turbidity, and samples were also taken to determine the protein concentration. Protein was determined by a modification of the Lowry method (Lowry et al, 1951); this involved addition of 10% SDS solution to the reaction mixture, at a ratio of 1:14 w/v, prior to addition of Folin-Ciocalteau reagent (Wang and Smith, 1975).

Stenotrophomonas maltophilia VUN 10,010 was tested for its ability to grow on a variety of anionic, cationic and nonionic surfactants supplied as a sole carbon source. Of these, only the Tween series could be utilized by strain VUN 10,010 as a growth substrate.

The agar diffusion method was used to determine the effects of surfactants on growth by observing zones of inhibition on peptone-glucose agar (Cserhali et al, 1991). The medium contained per litre: 5 g peptone, 5 g glucose, 0.5 g NaCl, 3 g beef extract, 20 g agar; the pH was adjusted to 7.0 and sterilized by autoclaving for 15 min at 121° C. Bacterial cultures were mixed with the molten medium (25 ml) to obtain an inoculum size of approximately 10⁸ cells ml⁻¹, and this was poured into a Petri dish. Once the cell-medium mixture had solidified, 7 mm diameter holes were made, and 150 μl of aqueous surfactant solutions was added (0.1-10.0 g l⁻¹). Growth inhibition zones were observed after 48-72 h at 30° C. The minimum inhibitory concentration (MIC) was taken to be the lowest surfactant concentration which resulted in clear zones of bacterial growth surrounding wells containing the surfactant solution.

The effect of surfactant on the growth of strain VUN 10,010 cultured on pyrene was determined by first incubating BSM (9 ml) containing pyrene (250 mg l⁻¹) overnight (30° C./175 rpm) to attain a saturated aqueous pyrene concentration. Surfactant (0.1 g l⁻¹) was then added to this medium, which was subsequently inoculated with pyrene-grown cells (ca. 10³ cells ml⁻¹). Growth was monitored by measuring protein concentration and viable cell numbers at regular intervals up to 10 days. Viable cell numbers were determined by most probable number (MPN) estimates (Collin et al, 1989). In well diffusion toxicity tests, the cationic surfactant, CTAB, was inhibitory at relatively low concentrations (0.01-0.05 g l⁻¹), and the anionic surfactant, SDS, had an MIC of 0.2 g l⁻¹. In contrast, the nonionic surfactants exerted little or no effect on microbial growth at concentrations up to 10 g l⁻¹. Igepal CA-630 caused some inhibition at concentrations of 5 and 10 g l⁻¹, and inhibition of growth by all surfactants was observed at 20 g l⁻¹.

### EXAMPLE 12

### Surfactant Critical Micelle Concentration and PAH Solubility

Based on these results, nonionic surfactants were used in experiments designed to optimize conditions for the biodegradation of high molecular weight PAHs. Surfactant Critical Micelle Concentration (CMC) and PAH solubilizing properties were determined.

Experiments to evaluate surfactant CMC values were conducted at 30° C. using a duNouy ring tensitometer (Cambridge Instrument Company Limited, London, England). All glassware was cleaned with chromic acid solution, and the tensitometer ring was cleaned with acetone and heated to redness in a gas flame. Various concentrations of surfactant solutions were prepared in deionized water and BSM. Surfactant solutions were allowed to equilibrate for approximately 2 h before measurements were made. Measurements were an average of five readings (SD<0.3%). CMC value was taken from the plot of surface tension versus log surfactant concentration, as described by Kile and Chiou (1989). Surfactant CMC measurements were performed in BSM and in sterile deionized water at 30° C., and slightly different values were obtained for these two solutions; Brij 35, Igepal CA-630 and Tergitol NP-10 gave higher CMC values in BSM, while Triton X-100 and Tyloxapol gave higher CMC values in distilled water (data not shown).

The results are summarized in Table 5.

TABLE 5
Surfactant properties and PAH solubility.

| | mol. wt | | CMC^{b} | MSR^{c} | | | log Kₘ^{c} | | |
|---|---|---|---|---|---|---|---|---|---|
| Surfactant | (average) | HLB^{a} | (mM) | PYR | FA | BAP | PYR | FA | BAP |
| Brij 35 | 1,200 | 16.9 | 0.0625 | 0.0570 | 0.0871 | 0.0198 | 5.54 | 5.68 | 7.78 |
| Igepal CA-630 | 603 | 13.0 | 0.1078 | 0.0326 | — | — | 5.39 | — | — |
| Tergitol NP-10 | 652 | 13.1 | 0.0690 | 0.0336 | 0.0502 | 0.0118 | 5.71 | 5.56 | 7.43 |
| Triton X-100 | 625 | 13.5 | 0.2320 | 0.0352 | 0.0508 | 0.0113 | 5.84 | 5.64 | 7.49 |
| Tyloxapol | 4,500 | 12.9 | 0.0167 | 0.0820 | — | — | 6.60 | — | — |

^{a}Hydrophile-lipophile balance number. Data was obtained from the manufacturers.
^{b}The critical micelle concentration of each surfactant was determined in BSM at 3000.
^{c}The Molar Solubilization Ratio and micelle-phase/aqueous-phase partition coefficient were determined for pyrene (PYR), Fluoranthene (FA) and Benzo[a]pyrene (RAP) in BSM at 30° C..
—Not tested.

### EXAMPLE 13

### PAH Solubilization Experiments

PAHs (approximately 1 g l⁻¹) were added to a 10 ml solution of BSM plus surfactant in a 30 ml glass reaction vial, which was sealed with an aluminium cap fitted with a septum. Surfactant concentrations corresponded to levels above, below, and at the CMC, and replicates were set up to allow sampling in duplicate at different time intervals up to 72 h. Incubation was performed at 30° C. and 175 rpm. The entire vial contents was collected by syringe and expressed through a preconditioned membrane filter (0.22 μm, Sartorius AG, Germany) to remove solid-phase crystalline PAH while allowing solubilized PAH in the aqueous phase and micellar pseudophase to pass into sterile vials. The filter was preconditioned by purging the filter with a few mls of sample solution to allow sorption saturation of the internal surfaces of the syringe, filter and needle. The filtrates were extracted with dichloromethane (DCM), and the concentration of solubilized PAH was determined by GC-FID as described below.

The apparent solubility of fluoranthene, pyrene and benzo[a]pyrene was measured in the presence of the nonionic surfactants at concentrations below, above and at the CMC. The solubility of dibenz[a,h]anthracene and coronene was low, and their concentrations in the aqueous phase at surfactant CMC were below the detection limit of the gas chromatograph. However, their aqueous concentrations increased to GC-detectable levels at higher surfactant concentrations. Time course experiments revealed that PAH concentrations in the aqueous pseudophase equilibrated in 42 h; measurements were therefore taken after this period. As shown in FIG. 10, the solubility of pyrene, fluoranthene and benzo[a]pyrene in the presence of the five nonionic surfactants slightly increased with increasing surfactant concentrations below the CMC. However, surfactant concentrations above the CMC proportionally increased the apparent solubility of pyrene, fluoranthene and benzo[a]pyrene such that their liquid phase concentrations were 0.48-0.56 mM (700-fold increase), 0.73-0.81 mM (500-fold increase) and 0.16-0.18 mM (10⁴-fold increase), respectively, when the surfactant concentrations were 10.0 g l⁻¹. The aqueous solubilities of pyrene, fluoranthene and benzo[a]pyrene at 30° C. in the absence of surfactant were approximately 6.97×10⁻⁴ mM, 1.37×10⁻³ mM and 1.59×10⁻⁵ mM, respectively. The exception for pyrene was Tyloxapol (10 g l⁻¹), which only increased the apparent pyrene concentration in the aqueous pseudophase to 0.182 mM (260-fold increase).

The effectiveness of a particular surfactant in solubilizing a given PAH can be assessed by determining the molar solubilization ratio (MSR) and micelle/water partition coefficient (Kₘ). In our study, the concentrations of added PAHs were well in excess of aqueous saturation concentrations; therefore the MSR value of each compound can be obtained from the slope of the curve generated from a plot of PAH concentration versus surfactant concentration for surfactant concentrations above CMC (Edwards et al, 1991). There is a high correlation factor for all the curves shown in FIG. 10 (greater than 0.999, 0.998 and 0.995 for pyrene, fluoranthene and benzo[a]pyrene respectively), and the corresponding MSR values for pyrene, fluoranthene and benzo[a]pyrene are shown in Table 6. Fluoranthene had the highest MSR value, and benzo[a]pyrene the lowest.

Kₘ values can be determined from the measured MSR values according to the following equation (Edwards et al, 1991):

$${K}_{m} = \frac{1}{{V}_{W} {S}_{PAH , CMC}} ( \frac{MSR}{1 + MSR} )$$

^{S}PAH, CMC is the apparent solubility of the PAH (mol l⁻¹) at surfactant CMC. V_{W} is the molar volume of water at 30° C. ie. 0.018015 l mol⁻¹(Rogers and Mayhew, 1981). Log Kₘ values were determined for fluoranthene, pyrene and benzo[a]pyrene in the presence of each nonionic surfactant using the above equation, and are shown in Table 6. The comparative log Kₘ values for each PAH varied depending on the surfactant, with the only clear trend being the significantly higher values obtained for benzo[a]pyrene compared to fluoranthene and pyrene.

TABLE 6
The Influence of Surfactants on Maximum Specific Degradation Rates
of High Molecular Weight PAHs

| | Maximum Specific Degradation Rate (mg PAH degraded mg⁻¹ protein day⁻¹)^{a} | | | | | | |
|---|---|---|---|---|---|---|---|
| | | | Tergitol NP-10^{b} | | | Brij 35^{b} | |
| | No Surfactant | | 5 g l⁻¹ | | 10 g l⁻¹ | 5 g l⁻¹ | 10 g l⁻¹ |
| PAH | Single^{c} | Mixed^{d} | Single^{c} | Mixed^{d} | Single^{c} | Single^{e} | Single^{e} |
| Fluoranthene | 2.90 | 1.02 | 4.54 (+57%) | 1.11 (+9%) | 4.59 (+58%) | 3.71 (+28%) | 12.11 (+318%) |
| Chrysene | 4.49 | 1.42 | 6.44 (+43%) | 2.76 (+94%) | 4.16 (−7%) | 7.35 (+64%) | 7.93 (+77%) |
| Benzanthracene | 4.41 | 1.71 | 9.66 (+119%) | 3.25 (+90%) | 11.14 (+153%) | 7.18 (+63%) | 7.37 (+67%) |
| Benzo[a]pyrene | 6.21 | 1.43 | 6.73 (+8%) | 4.30 (+201%) | 7.90 (27%) | 5.84 (−6%) | 5.47 (−12%) |
| Dibenz[a, h]anthracene | 4.17 | 1.20 | 7.32 (+76%) | 2.89 (+140%) | 8.74 (110%) | 6.26 (+50%) | 7.20 (+73%) |
| Coronene | 0.75 | — | 0.98 (+31%) | — | 0.79 (+5%) | 0.79 (+5%) | 1.19 (+59%) |

^{a}S. maltophilia VUN 10,010 was used for all experiments.
^{b}The percentage change in the PAH maximum specific degradation rate due to surfactant addition is shown in parenthesis.
^{c}These FIGS. were calculated from the data collected from the experiment shown in FIG. 6.10.
^{d}These FIGS. were calculated from the data collected from the experiment shown in FIG. 6.12.
^{e}These FIGS. were calculated from the data collected from the experiment shown in FIG. 6.11.
—Not appreciable.

### EXAMPLE 14

### Biodegradation of Crystallized and Surfactant-Solubilized Pyrene

Preliminary PAH degradation experiments were conducted using crystalline or DMF-solubilized pyrene, and the results are shown in FIG. 11.

Crystallized or DMF-solublized pyrene (250 mg L⁼¹ final concentration) was added to sterile BSM (9 mL), with or without addition of Tergitol NP-10 (10 g L⁻¹). These broths were then inoculated with approximately 10³ cells mL⁻¹ of VUN 10,010 from a pyrene-BSM starter culture. Three types of controls were used: cultures containing no surfactant, uninoculated broth, and killed cell cultures (0.2% HgCl₂). All cultures were incubated at 30° C./175 rpm. Bacterial growth was determined by MPN estimates.

The degradation of pyrene crystals by VUN 10,010 was extremely slow in the absence of surfactant. However, the degradation rate of crystalline pyrene was significantly improved in the presence of Tergitol NP-10 (10 g l⁻¹). The rate of degradation of DMF-solubilized pyrene by VUN 10,010 was significantly greater than that of crystallized pyrene in both the presence and absence of surfactant, presumably due to the much higher surface area to mass ratio of the solubilized pyrene particles and consequent increased biovailability. The highest pyrene degradation rate was observed when Tergitol NP-10 (10 g l⁻¹) was added to cultures containing DMF-solubilized pyrene. In view of the effect of crystal surface area on PAH degradation rate, PAHs solubilized in DMF were used in all further experiments to ensure consistent PAH crystal surface area to mass ratios within and between experiments.

### EXAMPLE 15

### Degradation of High Molecular Weight PAH

In general, the overall degradation rate of presolublized pyrene was signficiantly improved by the addition of 5-10 g l⁻¹ nonionic surfactants, as shown in FIG. 12. The time required for VUN 10,010 to degrade 250 mg l⁻¹ of pyrene was reduced by 50% in the presence of Brij 35, Tergitol NP-10 and Tyloxapol, but at the same concentrations (5-10 g l⁻¹) Igepal CA-630 inhibited pyrene degradation. The growth of VUN 10,010 on pyrene as determined by protein analysis and MPN estimates was significantly improved by Brij 35, Tergitol NP-10 and Tyloxapol, but inhibited by Igepal CA-630 (5-10 g l⁻¹).

### EXAMPLE 16

### Mineralization of Pyrene by VUN 10,010

Pyrene mineralization by S. maltophilia VUN 10,010 was demonstrated by the addition of [¹⁴CO₂]pyrene to cultures containing 250 mg l⁻¹unlabelled pyrene at the subsequent evolution of ¹⁴CO₂. These results are shown in FIG. 13. The addition of 5 g L⁻¹ Brij 35 or Tergitol NP-10 significantly improved the pyrene mineralization rate. In all cultures, the total recovery of added ¹⁴C was close to 100%, this being composed of 70-75% ¹⁴CO₂, the remainder being recovered from the cell pellet (10%), most probably in the form of biomass, the aqueous phase (15-18%), and in DCM after it was used for culture extraction. The significantly improved pyrene-degrading performance of VUN 10,010 due to Brij 35 and Tergitol NP-10 prompted further investigation into the effect of these two surfactants on the degradation of other high molecular weight PAHs by this strain.

### EXAMPLE 17

### Effect of High Initial Bacterial Population

Tergitol NP-10 (5 and 10 g l⁻¹) was added to BSM broths each containing a single high molecular weight PAH. A high initial cell population (ca 2×10⁶ cells ml⁻¹) prepared from washed inocula was used, since there was no substantial growth of VUN 10,010 on high molecular weight PAH compounds in the PAH degradation tests described in Table 1. The overall rate of biodegradation of most PAHs was substantially improved in the presence of Tergitol NP-10. For a majority of the cultures, this was the result of both an increase in the maximum specific degradation rate, as summarized in Table 7, and a decrease in the time taken for PAH degradation to commence, as shown in FIG. 14.

TABLE 7
The Influence of Surfactants on Maximum Specific Degradation Rates
of High Molecular Weight PAHs

| | Maximum Specific Degradation Rate (mg PAH degraded mg⁻¹ protein day⁻¹)^{a} | | | | | | |
|---|---|---|---|---|---|---|---|
| | | | Tergitol NP-10^{b} | | | Brij 35^{b} | |
| | No Surfactant | | 5 g l⁻¹ | | 10 g l⁻¹ | 5 g l⁻¹ | 10 g l⁻¹ |
| PAH | Single^{c} | Mixed^{d} | Single^{c} | Mixed^{d} | Single^{e} | Single^{e} | Single^{e} |
| Fluoranthene | 2.90 | 1.02 | 4.54 (+57%) | 1.11 (+9%) | 4.59 (+58%) | 3.71 (+28%) | 12.11 (+318%) |
| Chrysene | 4.49 | 1.42 | 6.44 (+43%) | 2.76 (+94%) | 4.16 (−7%) | 7.35 (+64%) | 7.93 (+77%) |
| Benzanthracene | 4.41 | 1.71 | 9.66 (+119%) | 3.25 (+90%) | 11.14 (+153%) | 7.18 (+63%) | 7.37 (+67%) |
| Benzo[a]pyrene | 6.21 | 1.43 | 6.73 (+8%) | 4.30 (+201%) | 7.90 (27%) | 5.84 (−6%) | 5.47 (−12%) |
| Dibenz[a, h]anthracene | 4.17 | 1.20 | 7.32 (+76%) | 2.89 (+140%) | 8.74 (110%) | 6.26 (+50%) | 7.20 (+73%) |
| Coronene | 0.75 | — | 0.98 (+31%) | — | 0.79 (+5%) | 0.79 (+5%) | 1.19 (+59%) |

^{a}S. maltophilia VUN 10,010 was used for all experiments
^{b}The percentage change in the PAH maximum specific degradation rate due to surfactant addition is shown in parenthesis.
^{c}These FIGS. were calculated from the data collected from the experiment shown in FIG. 6.10.
^{d}These FIGS. were calculated from the data collected from the experiment shown in FIG. 6.12.
^{e}These FIGS. were calculated from the data collected from the experiment shown in FIG. 6.11.
—Not appreciable

Benzo[a]pyrene was the exception, since the increase in the amount of this PAH degraded over the incubation period was largely due to a significant decrease in the degradation lag period. For all PAHs, an increase in Tergitol NP-10 concentration from 5 g l⁻¹ to 10 g l⁻¹ did not significantly improve the PAH degradation profile. Chrysene and coronene were the only PAHs which did not show improved PAH degradation rate in the presence of surfactants. Similar results were obtained when Brij 35 (5 g l⁻¹ and 10 g l⁻¹) was used. PAH concentrations over the time course period were not significantly different between the uninoculated controls and the inoculated controls containing both HgCl₂ and surfactant.

DCM extracts of samples taken from cultures inoculated with high cell numbers and containing benzo[a]pyrene, and the killed cell control, were further analysed by HPLC. These HPLC profiles, shown in FIG. 15, demonstrated the appearance of three compounds during incubation of the inoculated culture containing surfactant. Compound (I) was the only compound detected after 28 days, and its peak area decreased during the remainder of the incubation period; this was accompanied by an increase in peak areas of compounds (II) and (III). The identity of these compounds was not determined. No peaks other than that pertaining to benzo[a]pyrene were detected by HPLC in the killed cell control during incubation. These data support the contention that benzo[a]pyrene was degraded into other compounds by VUN 10,010.

### EXAMPLE 18

### Biodegradation of a Mixture of PAHs in the Presence of Surfactant

PAH-contaminated soils usually contain a mixture of different PAHs, which in many cases changes the degradation profile of each PAH from that observed when they are degraded as single PAH compounds. This is usually due to preferential substrate utilization and inhibitory effects on cell viability and growth caused by the greater toxicity of some PAHs.

The potential of strain VUN 10,010 for treating PAH-contaminated sites was further evaluated by investigating its ability to degrade a PAH mixture, comprising PAHs with three- to seven-benzene rings, in the presence of Tergitol NP-10 (5 g l⁻¹). A low initial cell population of around 9×10³ cells ml⁻¹ was used.

As shown in FIG. 16, Stenotrophomonas maltophilia VUN 10,010 was able to grow on the PAH mixture presumably due to the use of pyrene as a growth substrate. Although the specific growth rate was found to increase from 0.010 h⁻¹ in the absence of surfactant to 0.013 h⁻¹ in the presence of Tergitol NP-10, the biomass yield was not affected by the surfactant. The overall degradation rate of each PAH in the PAH cocktail was substantially improved by the addition of Tergitol NP-10. For most of the PAHs, this was attributable to a decrease in the degradation lag period and an increase in the maximum specific degradation rate, as shown in FIG. 14 and Table 6. The lag period and specific degradation rate for pyrene could not be measured using the data collected, due to its rapid consumption. Strain VUN 10,010 was able to degrade the PAHs concurrently. However, the maximum specific degradation rate of each PAH was less than that observed when these compounds were present in BSM as a single compound.

### EXAMPLE 19

### Degradation of PAHs in Soil in the Presence of Surfactant

The ability of the microorganisms of the invention to mineralize PAHs in soil was examined. Soil spiked with a mixture of PAHs was inoculated with 2% (w/w) Tergitol NP-10 together with a high initial population of either bacterial consortium VUN 10,009, S. maltophilia VUN 10,010, or P. janthinellium VUO 10,201, or co-cultures of bacteria plus fungus (Co-cultures A and B, as described in Example 6). Inocula of 10⁶ cell g⁻¹ soil for bacteria and 25 mg wet weight of mycelium g⁻¹ soil were used, and all cultures were incubated at 25° C. in the dark. The results are shown in FIG. 17. All of the cultures were able to degrade PAHs in the presence of the surfactant, in a similar fashion to that observed in Example 6, but Tergitol enhanced the rate of degradation.

### EXAMPLE 20

### Degradation of Other Organic Compounds

Using methods similar to those described above, we have shown that a variety of other aromatic and non-aromatic organic compounds can be degraded by the organisms of the invention in the absence of surfactants, and in particular that alkanes, halogenated and non-halogenated aromatic compounds and nitro-substituted aromatic compounds can act as sole carbon source for these organisms. The results are shown in Table 8.

TABLE 8
Growth of PAH-Degrading Bacteria
On A Variety Of Carbon Sources

| | Growth^{a} | | | |
|---|---|---|---|---|
| | VUN | VUN | VUN | VUN |
| C-source^{b} | 110,010 | 110,011 | 110,012 | 110,013 |
| Succinic acid | + | + | + | + |
| p-hydroxybenzoic acid | + | + | + | + |
| Maleic acid | + | + | + | + |
| Pyruvate | + | + | + | + |
| Protocatechuic acid | + | + | + | + |
| Catechol | + | + | + | + |
| D-panthothenic acid | + | + | + | + |
| Gentisic acid | + | + | + | + |
| Cinnamic acid | + | − | − | − |
| Benzoic acid | + | + | + | + |
| Salicylic acid | + | + | + | + |
| Phthalic acid | + | + | + | + |
| Tween 20 | + | + | + | + |
| Tween 80 | + | + | + | + |
| 2,5-dichlorophenol | + | + | + | + |
| 2,4,5-trichloro-phenol | + | + | + | + |
| 2,3,5,6-tetrachloro-phenol | + | + | + | + |
| Pentachlorophenol | + | − | + | − |
| 4-nitrophenol | − | − | − | − |
| 4-chlorophenol | + | − | + | + |
| Benzene^{c} | + | + | + | + |
| Toluene^{c} | + | + | + | − |
| n-hexadecane^{c} | + | + | + | + |
| n-octane^{c} | + | + | + | + |
| Nitrobenzene^{c} | + | − | + | − |

^{a}Growth was determined by visual observation of turbidity of culture media compare to uninoculated control.
^{b}Substrate concentration supplied to media was 50 mg I⁻¹.
^{c}Substrates were supplemented in vapor phase.

### DISCUSSION

Because of their environmental persistence, the focus of PAH research in recent years has been on the degradation of high molecular weight PAHs. This has resulted in the isolation of a number of microorganisms that can mineralise and grow on four-benzene ring PAHs as a sole carbon and energy source (Bouchez et al, 1995; Heitkamp et al, 1988; Juhasz et al, 1997; Kästner et al, 1994; Mueller et al, 1990; Walter et al, 1991; Weissenfels et al, 1990). Some of these isolates have been used to identify the biochemical pathways involved in the catabolism of four-benzene ring PAHs. However, microorganisms capable of degrading five-benzene ring PAHs have been more difficult to obtain.

Benzo[a]pyrene mineralisation by microorganisms has recently been demonstrated, but this compound has not previously been observed to support the growth of pure microbial cultures. Mineralisation of [¹⁴C]benzo[a]pyrene has been monitored after addition to soils and sediments; however, pure microbial cultures capable of degrading benzo[a]pyrene could not be isolated (Grosser et al, 1995; Grosser et al, 1991; Kanaly et al, 1997). Co-metabolic mineralisation of benzo[a]pyrene by pure microbial cultures has not been widely reported. However, Phanerochaete chrysosporium will mineralise benzo[a]pyrene in a medium containing another carbon substrate (Barclay et al, 1995; Bogan and Lamar, 1996; Bumpus et al, 1995; Sanglard et al, 1986). Bacteria have not been shown to mineralise co-metabolically benzo[a]pyrene in pure culture; however, pure cultures of Sp. paucimobilis EPA505 have been reported to mineralise benzo[a]pyrene in a phosphate buffer when a high population of resting cells was used. Such conditions were required because strain EPA505 could not grow on this PAH as a sole carbon and energy source. Strain EPA505 could only grow in the presence of benzo[a]pyrene when another growth-supporting PAH was present, but, since the mineralisation of benzo[a]pyrene by EPA505 was not tested under co-metabolic conditions, it is not known if EPA505 used benzo[a]pyrene co-metabolically as a carbon and energy source.

We have now demonstrated that axenic cultures of bacterial consortium VUN 10,009, S. maltophilia VUN 10,010 and P. janthinellum VUO 10,201 can degrade a number of tetracyclic and pentacyclic PAHs, including chrysene, dibenz[a,h]anthracene and benzo[a]pyrene, when present as a sole carbon and energy source. However, under such conditions these organisms cannot grow on the five-benzene ring PAHs, and were unable to effect significant mineralisation of benzo[a]pyrene.

Our isolates, S. maltophilia VUN 10,010 and bacterial consortium VUN 10,009, could co-metabolically mineralise benzo[a]pyrene in pure culture when pyrene served as the growth substrate. The degradation of at least a portion of the benzo[a]pyrene to CO₂ by VUN 10,010 and the bacterial consortium points to the existence of a catabolic pathway from which the bacteria may obtain carbon and generate energy from this PAH. The reason why benzo[a]pyrene alone cannot support the growth of VUN 10,010 and VUN 10,009 is not clear, but it may be due to the poor stimulation of PAH-catabolic enzyme synthesis by benzo[a]pyrene or its degradation products. The addition of pyrene to BSM and benzo[a]pyrene may compensate for this lack of enzyme synthesis via a positive analogue effect exerted by pyrene or its degradation products on the induction of benzo[a]pyrene catabolic enzymes (Boldrin et al, 1993), or because pyrene and benzo[a]pyrene may share similar catabolic pathways which are stimulated by pyrene in our bacterial strains.

The use of fungal-bacterial co-cultures to degrade PAHs has not previously been reported. We have shown that the co-culture of Penicillium janthinellum VUO 10,201 and bacterial consortium VUN 10,009 was able to mineralise and grow on benzo[a]pyrene as a sole carbon and energy source. Higher benzo[a]pyrene mineralisation and degradation rates were achieved when P. janthinellum VUO 10,201 was co-cultured with S. maltophilia VUN 10,010. This result is surprising, since VUN 10,010 was isolated from a different site to VUO 10,201, indicating that fungal-bacterial cooperative mineralisation of benzo[a]pyrene is not restricted to species isolated from the same site, and hence does not result from selective pressures. Microbial growth on dibenz[a,h]anthracene as a sole carbon and energy source has not previously been reported, yet our fungal-bacterial co-cultures were able to grow on this compound as a sole carbon source in BSM. This suggests that these co-cultures can also mineralise dibenz[a,h]anthracene.

The finding that neither P. janthinellum, S. maltophilia nor the bacterial consortium can independently mineralise or grow on benzo[a]pyrene as a sole carbon and energy source suggests that a mutually dependent relationship exists between the two organisms during PAH degradation in co-culture. Our HPLC data showed the accumulation of different benzo[a]pyrene degradation products in axenic P. janthinellum cultures (compound I) and bacterial cultures (compounds II, III and IV), which were further substantially or totally degraded only in the co-cultures. The cooperative catabolic relationship between the fungus and the bacteria was authenticated by the appearance and disappearance of a degradation compound compound V, which was not detected in the axenic cultures. These degradation products have not yet been identified. However, fungi have previously been reported to oxidise benzo[a]pyrene to trans-dihydrodiols at the 4,5, 7,8 or 9,10 positions (Cerniglia and Gibbons, 1980; Cerniglia et al, 1979; Launen et al, 1995), and a P. janthinellum strain has been reported to oxidise benzo[a]pyrene to 9-hydroxy-benzo[a]pyrene. It therefore appears likely that compound I is a mono- or dehydroxylated metabolite of PAH. For bacteria, Mycobacterium sp. RJGII-135 has recently been reported to oxidise benzo[a]pyrene initially to cis-dihydrodiols in the 4,5, 7,8 or 9,10, positions and then to 4,5-chrysene-dicarboxylic acid, 7,8-dihydro-pyrene-7-carboxylic acid or 7,8-dihydro-pyrene-8-carboxylic acid, respectively (Schneider et al, 1996).

The cooperative catabolic route by which benzo[a]pyrene is degraded in the co-cultures is not clear at this stage. However, a number of previous reports have suggested that PAH degradation in nature is a consequence of sequential breakdown by fungi and bacteria, with the fungi performing the initial PAH oxidation step (Meulenberg et al, 1997; Sack et al, 1997). Without wishing to be limited by any proposed mechanism, we believe that some of our data are consistent with the hypothesis that the fungi perform a major role in the initial oxidation of benzo[a]pyrene. For example, benzo[a]pyrene degradation and mineralisation in soil and in BSM containing benzo[a]pyrene and pyrene by axenic bacterial cultures commences only after considerable lag periods; such degradation lag periods are absent in axenic P. janthinellum cultures in soil. These degradation and mineralisation lag periods are also absent in co-cultures; this is not expected to be the case if bacteria are responsible for initiating benzo[a]pyrene oxidation. Moreover, a P. janthinellum degradation product, compound I, was the first degradation product to appear in co-cultures containing benzo[a]pyrene as the sole carbon and energy source. As mentioned earlier, compound I is most likely a mono- or dihydroxylated PAH metabolite, and such compounds have been proposed to be more easily degraded substrates for bacteria compared to the parent PAH (Heitkamp et al, 1988). This compound in fungal-bacterial co-cultures may act as a bacterial growth substrate which may be used as the only carbon source for the bacteria, which thus rely on the fungus alone to initially oxidise benzo[a]pyrene; alternatively or the bacteria may use the compound as a substrate for cometabolic benzo[a]pyrene mineralisation in a similar way to their use of pyrene for cometabolic benzo[a]pyrene mineralisation, with initial benzo[a]pyrene oxidation in the latter case being performed by both the fungus and the bacteria. P. janthinellum clearly profits from its relationship with the bacteria, since it is able to grow in BSM containing benzo[a]pyrene; growth does not occur if the bacteria are absent. Growth of the fungi is most likely attributable to a bacterial metabolic product which can serve as a fungal growth substrate; this hypothesis is supported by the inability of axenic P. janthinellum cultures to degrade or grow on pyrene as a sole carbon source, while fungal growth occurs in the presence of pyrene when pyrene-degrading bacteria are added to the culture.

The potential drawback with field use of co-cultures is that the soil environment, being a heterogeneous mix of organics and microbes, may destabilise the co-culture, possibly resulting in poor degradation rates, failure to degrade some PAHs, and/or the production of toxic, water soluble intermediates. Our results suggest that the co-cultures of this invention may avoid these disadvantages, since we have observed faster and more extensive degradation of low and high molecular weight PAHs, especially benzo[a]pyrene mineralisation, in authentic PAH-contaminated soil using a combined Penicillium and Stenotrophomonas inoculum compared to single organism inocula and to the indigenous microflora. A small amount of benzo[a]pyrene was mineralised in the authentic PAH-contaminated soil inoculated only with P. janthinellum. Since the fungus alone cannot mineralise this PAH, it appears that the indigenous bacteria and P. janthinellum were cooperatively mineralising benzo[a]pyrene. This is consistent with the finding that benzo[a]pyrene mineralisation was not observed in sterile PAH-contaminated soil inoculated only with P. janthinellum. As with the liquid culture experiments, there was a striking decrease in the mutagenic potential of the contaminated soil inoculated with the co-culture compared to single organism inocula. This is probably due to the higher degradation rates and to the formation of less toxic degradation byproducts eg CO₂ by the co-culture, compared to cultures inoculated with a single organism.

Our data have revealed the extraordinary ability of the fungal-bacterial co-cultures to grow on five-benzene ring PAHs, and to mineralise benzo[a]pyrene, as a sole carbon and energy source. Inoculation of these co-cultures into authentic PAH-contaminated soil demonstrated their superiority in the degradation and mineralisation of high molecular weight PAHs, and consequent reduction in mutagenicity of these compound soils, compared to axenic inocula and the indigenous microflora. These results indicate that the isolates of this invention are useful for the complete bioremediation of PAH-contaminated sites.

The scarcity of microbial isolates able to degrade high molecular weight PAHs most likely accounts for the limited amount of research into the use of surfactants for degrading these PAHs. The strain VUN 10,010 isolated in this work was capable of degrading PAH compounds containing up to five-benzene rings as sole carbon and energy sources, although substantial microbial growth was only observed in the presence of pyrene and low molecular weight PAHs. There was only a slight increase in protein concentration in the high molecular weight PAH degradation tests described in Table 1, indicating that growth was poor or limited. Pyrene mineralization by VUN 10,010 was demonstrated by the production of ¹⁴CO₂ from cultures containing labelled pyrene, but the extent of fluoranthene, chrysene, benz[a]anthracene, dibenz[a,h]anthracene and benzo[a]pyrene degradation by VUN 10,010 cannot be deduced from our results. HPLC analysis identified the production of three compounds in inoculated cultures containing benzo[a]pyrene which were not detected in the control cultures; in fact, only the benzo[a]pyrene peak was identified in the control cultures during 49 days incubation. The appearance of compounds I, II and III suggests that VUN 10,010 is at least transforming benzo[a]pyrene into other compounds, while compound I was itself transformed in the later stages of the incubation. Cometabolic transformation is not likely in this case, since benzo[a]pyrene was the only carbon and energy source in the culture.

It is generally known that nonionic surfactants are less toxic to microorganisms than cationic or anionic surfactants (Rouse et al, 1994; Volkering et al, 1995), and this was also true for strain VUN 10,010. This strain was able to use the Tween series of surfactants as growth substrates, which is undesirable since it lowers the surfactant concentration and, if used as a preferred carbon source, could result in the repression of PAH-degrading ability of the isolate. A proportional increase in PAH solubility was observed when surfactant concentrations were increased above CMC. The apparent solubility of pyrene and fluoranthene in BSM was increased by over 250-fold and the apparent solubility of benzo[a]pyrene was increased by a factor of 10³-10⁴ at a surfactant concentration of 10 g l⁻¹. It has previously been observed that the apparent solubility of phenanthrene was greater when more lipophilic surfactants, ie. surfactants of lower hydrophile-lipophile balance number (HLB) were used (Guerin and Jones, 1988). However, no correlation between surfactant HLB value and the apparent PAH solubility could be drawn from our results.

Lower MSR and higher log Kₘ values were observed for benzo[a]pyrene relative to those measured for fluoranthene and pyrene. Compared to the latter two PAHs, this indicates that, on a molar basis, less benzo[a]pyrene was incorporated into the surfactant micelles from the nonpolar pseudophase, and that a greater proportion of the total amount of benzo[a]pyrene in the micelle/aqueous pseudophases was embodied in the micelles. This result can be attributed to the much higher hydrophobicity of benzo[a]pyrene compared to that of fluoranthene and pyrene. The MSR and log Kₘ values which we obtained for pyrene using Tergitol NP-10 and Triton X-100 are comparable to those obtained by Edwards et al (1991), the slight differences most likely being attributable to our measurements being performed in an ionic solution at a higher temperature (viz. 30° C.) compared to 25° C. Ghosh et al (1995) reported MSR and log Kₘ values for benzo[a]pyrene in the presence of Triton X-100 to be 1.5×10⁻² and 7.56 respectively. These results for benzo[a]pyrene are slightly different to the values we obtained, but this may also be due to a difference in solubilization medium and temperature; Ghosh et al (1995) did not specify either of these parameters. Besides the work of Ghosh et al (1995), there are no other reports which determine the molar MSR and log Kₘ values for fluoranthene and benzo[a]pyrene in the presence of the nonionic surfactants used in this study. These values are important for the proper modeling of PAH partitioning in contaminated soils following surfactant addition (Aronstein et al, 1991; Edwards et al, 1991).

Surfactants have previously been observed to reduce or inhibit biodegradation because of surfactant toxicity to the bacteria (Laha and Luthy, 1992; Lin et al, 1995; Tiehm, 1994) or increased PAH toxicity due to its higher apparent solubility (Thibault et al, 1996). Our results show that microbial growth and pyrene degradation by VUN 10,010 was enhanced by the addition of Brij 35, Tergitol NP-10, Triton X-100 and Tyloxapol at concentrations up to 10 g l⁻¹. The specific growth rate on pyrene in the presence of Brij 35 or Tergitol NP-10 at 10 g l⁻¹ (0.015 h⁻¹) was 67% greater than the growth rate in the absence (0.009 h⁻¹) of these two nonionic surfactants. Igepal CA-630 at 5 g l⁻¹ was inhibitory to pyrene degradation which correlates to its growth-inhibitory effect at this concentration on glucose-grown VUN 10,010. Enhanced PAH transformation was also observed when Brij 35 and Tergitol NP-10 were used in strain VUN 10,010 cultures containing high numbers of washed cells and a single high molecular weight PAH, or in cultures inoculated with low numbers of washed cells and containing mixed PAHs. The maximum specific PAH degradation rate for most PAH compounds supplied as single or mixed PAH substrates was increased by surfactant addition. The disappearance of coronene was an exception, since no significant difference in its concentration was detected between the control and experimental cultures in the single PAH experiments, indicating that VUN 10,010 is unable to degrade this compound as a sole carbon and energy source under the experimental conditions used. However, significant amounts of coronene disappeared in the mixed PAH substrate cultures, suggesting cometabolic transformation of this compound. Tiehm (1994) observed that biomass production was dependent on the amount of surfactant added to Mycobacterium cultures containing a mixture of three- and four-ring PAHs; the biomass yield changed from around 20 mg l⁻¹ in the absence of surfactant to 60 mg l⁻¹ in the presence of 4 mM Sapogenat T-300. In a PAH mixture containing up to seven-ring PAHs, we found that, although the specific growth rate of the strain VUN 10,010 increased by 30% from 0.010 h⁻¹ (in the absence of surfactant) to 0.013 h⁻¹ (in the presence of 5 g l⁻¹ Tergitol NP-10), the cell yield (around 83 mg l⁻¹) was largely independent of the presence or absence of Tergitol NP-10. These results suggest that substrate solubility rather than biomass yield limited the PAH utilization rate of strain VUN 10,010 grown in PAH mixtures.

Without wishing to be limited by any proposed mechanism, we believe that surfactant enhancement of PAH biodegradation results from enhancement of the PAH mass transfer rate into the aqueous phase by increasing the solid phase dissolution rate plus PAH incorporation into micelles; the dynamic exchange of PAH molecules between the micelle and the aqueous phase replenishes the supply of PAH compounds more quickly into the aqueous pseudophase as they are degraded (Liu et al, 1995; Volkering et al, 1996). The surfactant-enhanced PAH degradation and growth rates of the strain VUN 10,010 suggests that the degradation performance of our surfactant-free cultures was restricted in part by the PAH dissolution rate, supporting the above hypothesis. However, two aspects of our results alone cannot be explained in terms of PAH dissolution rate alone. Firstly, our results show that surfactants reduce the PAH degradation lag period. An enhanced PAH dissolution rate cannot explain this observation, since PAH utilization during the degradation lag period is very low, if at all. Secondly, the degradation rates of phenanthrene and fluorene were substantially slower than that of pyrene in the mixed PAH control cultures which did not contain surfactant. Since both of the former PAHs have higher dissolution rates than pyrene, this suggests that the rate-limiting step for biodegradation of phenanthrene and fluorene in the PAH mixture is not their dissolution rate into the aqueous pseudophase, but it is associated with the rate at which strain VUN 10,010 can catabolise these two compounds. However, a significant improvement in phenanthrene and fluorene degradation rates was observed when surfactant was added to these cultures, which should not be possible if surfactants only enhance the PAH mass transfer rate into the aqueous pseudophase. These observations suggest that there may be mechanisms involved in the surfactant enhancement of high molecular weight PAH degradation in addition to an increased PAH dissolution rate. This is supported by recent studies which found that PAH biodegradation in the presence of surfactants was greater than predicted by models which assumed that microorganisms did not have direct access to micellized PAHs (Guha and Jaffé, 1996; Liu et al, 1995). Other proposed mechanisms include direct access of the microorganism to micelle-bound PAHs via complex transport mechanisms, adherence of the microorganisms to hydrophobic surfaces (Jimenez and Bartha, 1996) and membrane fusion of surfactant micelles (Miller and Bartha, 1989).

We have found that S. maltophilia sp. VUN 10,010 can degrade or transform high molecular weight PAHs containing up to seven-benzene rings in the presence of non-ionic surfactants. Furthermore, the PAH degradation rate is substantially improved when surfactants are present in single or mixed PAH cultures and strain VUN 10,010 can grow rapidly on a mixture of high molecular weight PAHs which serve as sole carbon and energy sources. Although soil flushing technologies have recently been shown to be highly successful when surfactant solutions are used to solubilize recalcitrant high molecular weight PAHs from weathered PAH contaminated sites (Chuchhill et al, 1995; Joshi and Lee, 1996; Yeom et al, 1996), there have been no previous reports of microbial isolates capable of degrading the leachates from such operations. When bioreactors containing strain VUN 10,010 or similar isolates are used in conjunction with soil-flushing techniques, it may be possible to effectively decontaminate PAH-polluted soils which would be highly recalcitrant to degradation using other bioremediation techniques.

### REFERENCES

Altschul, S. F., G. Warren, M. Webb, E. W. Myers and D. J. Lipman. 1990. Basic local alignment search tool. J. Mol. Biol. 215, 4013-410.

Anderson, B. E. and Henrysson, T. Appl. Microbiol. Biotechnol., 1996 46 647-652.

Aronstein, B. N., Calvilllo, Y. M. and Alexander M. Environ. Sci. Technol., 1991 25 1728-1731.

Barclay, C. D., Farquhar, G. F. and Legge, R. L. Applied Microbiology and Biotechnology, 1995 42 958-963.

Bezalel, L., Hader, Y., Fu, P. P., Freeman, J. P. and Cerniglia, C. E. Appl. Environ. Microbiol., 1996 62 2554-2559.

Bogan, B. W. and Lamar, R. T. Appl. Environ. Microbiol., 1996 62 1597-1603.

Boldrin, B., Tiehm, A. and Fritzshche, C. 1993. Appl. Environ. Microbiol., 1993 59 1927-1930.

Bouchez, M., Blancher, D. and Vandecasteele, J-P. Appl. Microbiol. Biotechnol., 1995 43 156-164.

Brodkorb, T. S. and R. L. Legge. Appl. Environ. Microbiol., 1992 58 3117-3121.

Bumpus, J. A. Appl. Environ. Microbiol., 1989 55 154-158.

Bumpus, J. A., Tien, M., Wright, D. and Aust, S. D. Science, 1985 228 1434-1436.

Bury, S. J. and Miller, C. A. Environ. Sci. Technol., 1993 27 104-110.

Cerniglia, C. E. Biodegradation, 1992 3 351-368.

Cerniglia, C. E. and Gibson, D. T. J. Biol. Chem., 1979 254 12174-12180.

Cerniglia, C. E. and Gibson, D. T. J. Biol. Chem., 1980 255 5159-5163.

Churchill, P. F., Dudley R. J. and Churchill, S. A. 1995. Waste Manage., 1995 15 371-377.

Collins, P. J., Kotterman, M. J. J., Field, J. A. and Dobson, A. D. W. Appl. Environ. Microbiol., 1996 62 45634567.

Collins, C. H., Lyne, P. M. and Grange, J. M. Microbiological methods. 6th edition. Butterworths, London, 1989

Cserhali, T., Illes, Z. and Nemes, I. Appl. Environ. Biotechnol., 1991 35 115-118.

Deschenes, L., Lafrance, P., Villenuve, J-P. and Samson R. In: R. E. Hinchee, F. J. Brockman, and C. M. Vogel (eds.), Microbial processes for bioremediation. Battelle Press, Columbus, Richland, 1995

Edwards, D. A., Luthy, R. G. and Liu, Z. 1991. Environ. Sci. Technol., 1991 25 127-133.

Fedorak, P. H., Fought, J. M. and Westlake, D. W. S. Water Res., 1982 16 1285-1290.

Fought, J. M., Gutnick, D. L. and Westlake, D. W. S. Appl. Environ. Microbiol., 1989 55 3642.

Ganeshalingam, S., Legge, R. L. and Anderson, W. A. Process Saf. Environ. Prot., 1994 72 247-251.

Ghosh, M. M., Yeom, I. T., Shi, Z., Cox, C. D. and Robinson, K. G. In: R. E. Hinchee, F. J. Brockman, and C. M. Vogel (eds.), Microbial processes for bioremediation. Battelle Press, Columbus, Richland, 1995 15-23

Grimberg, S. J., Aitken, M. D. and Stringfellow, W. T. Wat. Sci. Tech., 1994 30 23-30.

Grimberg, S. J., Stringfellow, W. T. and Aitken, M. D. Appl. Environ. Microbiol., 1996 62 2387-2392.

Grosser, R. J., Warshawsky, D. and Vestal, J. R. Appl. Environ. Microbiol., 1991 57 3462-3469.

Grosser, R. J., Warshawsky, D. and Vestal, J. R. Environmental Toxicology and Chemistry, 1995 14 375-382.

Guerin, W. F. and Jones G. E. Appl. Environ. Microbiol., 1988 54 937-944.

Guha, S. and Juffé, P. R. Environ. Sci. Technol., 1996 30 605-611.

Heitkamp, M. A., Freeman, J. P., Miller, D. W. and Cerniglia, C. E. Appl. Environ. Microbiol., 1988 54 2556-2565.

Higgins, D. C. and P. M. Sharp. 1988. CLUSTAL: a package for performing multiple alignment on a mini-computer. Gene 73, 237-244.

Jimenez, I. Y. and Bartha, R. Appl. Environ. Microbiol., 1996 62 2311-2316.

Joshi, M. M. and Lee, S. Energy Sources, 1996 18 291-301.

Juhasz, A. J., Britz, M. L. and Stanley, G. A. Biotechnol. Lett., 1996 18 577-582.

Juhasz, A. L., Britz, M. L. and Stanley, G. A. J. Appl. Microbiol., 1997 83 189-198.

Kanaly, R., Bartha, R., Fogel, S. and Findlay, M. Appl. Environ. Microbiol., 1997 63 4511-4515.

Kästner, M., Breuer-Jammali, M. and Mahro, B. Appl. Environ. Microbiol., 1994 41 267-273.

Keith, L. H. and Telliard, W. A. Environ. Sci. Technol., 1979 13 416-423.

Kiehlmann, E., Pinto, L. and Moore, M. Can. J. Microbiol., 1996 42 604-608.

Kile, D. E. and Chiou, C. T. Environ. Sci. Technol., 1989 23 832-838.

Kiyohara H., Nagoa K. and Yana K. Appl. Environ. Microbiol., 1982 43 454-457.

Laha, S. and Luthy, R. G. Environ. Sci. Technol., 1991 25 1920-1930

Launen, L., Pinto, L., Wiebe, C., Kiehlmann, E. and Moore, M. Can. J. Microbiol., 1995 41 477-488.

Lowry, O. H., Rosebrough, N. J., Farr, A. L. and Randall, R. J. J. Biol. Chem., 1951 193 265-275.

Liu, Z., Jacobson, A. M. and Luthy, R. G. 1995. Appl. Environ. Microbiol., 1995 61 145-151.

McElroy, A. E., Farrington J. W. and Teal J. M. Appl. Environ. Microbiol., 1989 55 269-274.

Mahaffey, W. R., Compeau, G., Nelson, M. and Kinsella, J. Water Environ. Technol., 1991 3 83-88.

Maron, D. M. and Ames, B. N. Mut. Res., 1983 113 173-215.

Meulenberg, R., Rijnaarts, H. H. M., Doddema, H. J. and Field, J. A. FEMS Microbiol. Lett., 1997 152 4549.

Morehead, N. R., Eadie, B. J., Lake, B., Landrum, P. D. and Berner, D. [REFERENCE DETAILS REQUIRED] 1986

Mueller, J. G., Chapman, P. J., Blattmann, B. O. and Pritchard, P. H. Appl. Environ. Microbiol., 1990 56 1079-1086.

Pothuluri, J. V., Selby, A., Evans, F. E., Freeman, J. P. and Cerniglia, C. E. Can. J. Bot., 1994 73 S1025-S1033.

Rogers, G. F. C. and Mayhew, Y. R. Thermodynamic and transport properties of fluids. 3rd edition. Basil Blackwell, Oxford, 1981

Rouse, J. D., Sabatini, D. A., Suflita, J. M. and Harwell, J. H. Crit. Rev. Environ. Sci. Technol., 1994 24 325-370.

Sack, U., Heinze, T. M., Deck, J., Cerniglia, C. E., Martens, R., Zadrazl, F. and Fritsche, W. Appl. Environ. Microbiol., 1997 63 3919-3925.

Sanglard, D., Leisola, M. S. A. and Fiechter, A. Enzyme Microb. Technol., 1986 8 209-212.

Scheibenbogen, K., Zytner, R. G., Lee, H., Trevors, J. T. J. Chem. Technol. Biotechnol., 1994 59 53-59.

Schneider, J., Grosser, R., Jayasimhulu, K., Xue, W. and Warshawsky, D. Appl. Environ. Microbiol., 1996 62 13-19.

Shuttleworth, K. L. and Cerniglia, C. E. Appl. Biochem. Biotechnol., 1995 54 291-302.

Sutherland, J. B. J. Ind. Microbol., 1992 9 53-62.

Thibault, S. L., Anderson, M. and Frankenberger JR. W. T. Appl. Environ. Microbiol., 1996 62 283-287.

Tiehm, A. Appl. Environ. Microbiol., 1994 60 258-263.

Volkering, F., Breure, A. M., Van Andel, J. G. and Rulkens, W. H. Appl. Environ. Microbiol., 1995 61 1699-1705.

Vyas, B. R. M., Bakowski, S., Sasek, V. and Matucha, M. FEMS Microbiology Ecology., 1994 14 65-70.

Walter, U., Beyer, M., Klein, J. and Rehm, H. J. Appl. Microbiol. Biotechnol., 1991 34 671-676.

Wang, C. and Smith, R. L. Anal. Biochem., 1975 63 414-417.

Weissenfels, W. D., Beyer, M. and Klein, J. Appl. Microbiol. Biotechnol., 1990 32 479-484.

Wilson, S. C. and Jones, K. C. Environmental Pollution, 1993 88 229-249.

Wright, A. S. Mutat Res., 1980 75 215-241.

Wunder T., Marr, J., Kremer, S., Sterner, O. and Anke, H. 1997 Arch. Microbiol., 1997 167 310-316.

Ye, D., Siddiqi, M. A., Maccubbin, A. E., Kumar, S. and Sikka, H. C. Environ. Sci. Technol., 1996 30 136-142.

Yeom, I. T., Ghosh, M. M. and Cox, C. D. Environ. Sci. Technol., 1996 30 1589-1595.

The claims defining the invention are as follows:

1. An isolated culture of Stenotrophomonas maltophilia, VUN 10,012, having Accession No. NM98/09530, or a mutant or derivative thereof having the ability to degrade high molecular weight polycyclic aromatic hydrocarbons (PAHs).

2. An agent for degrading PAHs, comprising a co-culture of Penicillium janthinellum VUO 10,201 and bacterial consortium VUN 10,009.

3. An agent according to claim 2, further comprising at least one microorganism selected from the group consisting of Stenotrophomonas maltophilia VUN 10,010 or VUN 10,012, Pseudomonas fluorescens VUN 10,011 and Burkholderia sp. VUN 10,013.

4. An agent according to claim 2, further comprising Stenotrophomonas maltophilia VUN 10,010 or VUN 10,012, Pseudomonas fluorescens VUN 10,011 and Burkholderia sp. VUN 10,013.

5. An agent for degrading PAHs, comprising a co-culture of Penicillium janthinellum VUO 10,201 and bacterial consortium VUN 10,009 or Stenotrophomonas maltophilia VUN 10,010.

6. A composition for degrading PAHs, comprising a co-culture of Penicillium janthinellum VUO 10,201 and bacterial consortium VUN 10,009 together with one or more carriers or excipients.

7. A composition according to claim 6, further comprising at least one microorganism selected from the group consisting of Stenotrophomonas maltophilia VUN 10,010 or VUN 10,012, Pseudomonas fluorescens VUN 10,011 and Burkholderia sp. VUN 10,013.

8. A composition according to claim 6, further comprising Stenotrophomonas maltophilia VUN 10,010 or VUN 10,012, Pseudomonas fluorescens VUN 10,011 and Burkholderia sp. VUN 10,013.

9. A composition for degrading PAHs, comprising a co-culture of Penicillium janthinellum VUO 10,201 and bacterial consortium VUN 10,009 or Stenotrophomonas maltophilia VUN 10,010.

10. A composition according to any one of claims 6 to 9, in the form of a spray, foam, liquid or in lyophilized form.

11. A method for degrading PAHs, comprising the step of exposing the PAHs to the isolated culture according to claim 1, an agent according to any one of claims 2 to 5, or a composition according to any one of claims 6 to 10.

12. A method according to claim 11, suitable for bioremediation of soils, sediments or liquids contaminated with PAHs.
